(19) United States
(12) Patent Application Publication
(10) Pub. No.: US 2026/0224485 A1
(43) Pub. Date: Aug. 6, 2026

(54) THERAPEUTIC COMPOSITIONS AND METHODS

(71) Applicant: SiSaf Ltd, Guildford Surrey (GB)

(72) Inventors: Roghieh Suzanne SAFFIE-SIEBERT, Guildford Surrey (GB); Flavia SUTERA, Guildford Surrey (GB)

(73) Assignee: SiSaf Ltd, Guildford Surrey (GB)

(21) Appl. No.: 19/148,585

(22) PCT Filed: Jan. 19, 2024
(86) PCT No.: PCT/GB2024/050156

§ 371 Date: Jul. 16, 2025

(30) Foreign Application Priority Data
Jan. 20, 2023 (GB) 2300911.1

(51) Int. Cl. A61K 9/1271; A61K 9/16; A61K 39/00; A61K 39/215

(52) U.S. Cl. CPC A61K 9/1271; A61K 9/1611; A61K 39/215; A61K 2039/53

(57) ABSTRACT

Disclosed is a composition comprising: particles comprising one or more metals and hydrolysable silicon; one or more lipids; and an active pharmaceutical ingredient (API). Also disclosed are related products, methods and uses.

### FIELD OF THE DISCLOSURE

The present disclosure concerns delivery vehicles for active pharmaceutical ingredients (“APIs”). More particularly, but not exclusively, this disclosure concerns a composition comprising: (i) particles comprising one or more metals and hydrolysable silicon; (ii) one or more lipids; and (iii) an active pharmaceutical ingredient (API). This disclosure especially concerns a composition comprising (i) particles comprising calcium (especially, Ca²⁺) dispersed throughout a hydrolysable silicon matrix; (ii) one or more lipids; and (iii) an API. The disclosure also concerns related products, medical uses, and methods.

### BACKGROUND

A need exists for improvements in vehicles for API delivery. This is required for advances in biomedical research to be fully translated into efficient, safe and cost-effective treatments.

Recent biological advances have provided insight into new classes of API for the prophylaxis and treatment of many diseases. One example of such a class is nucleic acids, especially mRNA, siRNA, tRNA and circular RNA (oRNA). By way of example, a variety of mRNA-based infectious disease vaccines have recently come to light. Messenger RNA (mRNA)-based cancer therapies have also now entered clinical development. Meanwhile, siRNA shows promise for the treatment of various genetic diseases and disorders.

However, with the promise of new classes of API comes the challenge of how to control API stability during storage. It must also be ensured that APIs actually reach and are taken up by relevant cells once they have been administered to a patient; it may be challenging to stabilise APIs while they are circulating in the body. Moreover there exists a need for tissue or cell targeting, so that an API can be delivered to the correct cells. Once a target cell is reached, then if the API is intended for delivery into the cell (rather than, for example, an API intended to act outside a cell, such as on a cell surface protein) there is also the challenge of how to ensure efficient API uptake by cells. Following such uptake, there is a need to ensure API stability in the cytoplasm for a sufficient time period so that it is able to exert sufficient biological effect.

In one approach, lipid nanoparticles, mainly comprising exogeneous cationic or ionisable lipids, have been used for the in vivo delivery of fragile APIs such as nucleic acids and especially mRNA. (As used herein, the terms “fragile APIs” and “reactive APIs” may be interchangeable and may refer to APIs which (i) have a half-life upon storage in aqueous solution at about 25° C. of at most one week, measurable by NMR or by GC-MS; and/or (ii) have a half-life in vivo of under about an hour, measurable by assay of a biological sample.) Cold-chain storage is required for fragile API-containing lipid nanoparticles, which limits their distribution and is not energy-efficient or cost-effective.

There remains a need for delivery vehicles with an increased capability for stabilising fragile APIs, both during storage (so that they can be stored at higher temperatures) and while they are circulating in the body (so that more API reaches target cells more quickly).

Meanwhile, some lipid nanoparticle-based delivery vehicles may display cytotoxicity. Commonly used lipid nanoparticles rely on exogeneous cationic lipid(s) (i.e., lipid(s) which has or have a net positive charge at a pH of about 7.4; also known as exotic cationic lipids) containing multiple amine groups. Although well-suited to the electrostatic loading of polyanionic nucleic acids (including various form of RNA) these amine-rich species may lead to cytotoxicity, immunogenicity, and non-specific tissue accumulation. Similar problems occur with other, polymer-based, amine-rich delivery systems. There exists a need for improved delivery vehicles which also have an acceptably low toxicity.

Preferably, new delivery vehicles would also be fully dispersible in an aqueous environment, to ensure ease of delivery, such as by injection in aqueous solution or an aqueous suspension.

The present disclosure seeks to mitigate the above-mentioned problems. Alternatively or additionally, the present disclosure seeks to provide an improved API delivery vehicle.

### SUMMARY OF THE DISCLOSURE

In a first aspect of the present disclosure, there is provided a composition comprising: (i) particles comprising one or more metals and hydrolysable silicon; (ii) one or more lipids; and (iii) an active pharmaceutical ingredient (API). Preferably, the composition comprises (i) particles comprising calcium (especially, Ca²⁺) dispersed throughout a hydrolysable silicon matrix; (ii) one or more lipids; and (iii) an API.

In a second aspect, there is provided a composition defined in accordance with the first aspect, for use in a method of preventing or treating a disease or disorder in a human subject.

In a third aspect, there is provided a method of preventing or treating a disease or disorder, comprising administering a prophylactically or therapeutically effective amount of a composition defined in accordance with the first aspect or the second aspect to a human subject in need thereof.

In a fourth aspect, there is provided the use of a composition defined in accordance with the first aspect or the second aspect, in the manufacture of a medicament for use in a method defined in accordance with the second aspect or the third aspect.

In a fifth aspect, there is provided a method for slowing the degradation of an API, comprising: (i) contacting one or more lipids with particles comprising one or more metals and hydrolysable silicon; and (ii) contacting the lipids and particles with the API. Preferably, the method comprises (i) contacting one or more lipids with particles comprising calcium (especially, Ca²⁺) dispersed throughout a hydrolysable silicon matrix; and (ii) contacting the lipids and particles with the API. The method may further comprise contacting the lipids and particles obtained in step (i) with one or more of tyrosine, NAD, quercetin and derivatives thereof, prior to the step of contacting the lipids and particles with the API.

### DESCRIPTION OF THE DRAWINGS

Embodiments of the present disclosure are now described by way of reference to the accompanying drawings in which:

FIG. 1 shows the manufacture of calcium- and silicon-containing particles in accordance with Example 1a.

FIGS. 2 and 3 show the results of transfection of rat muscle cells with delivery systems having calcium- and silicon-containing particles, compared to delivery systems having silicon-containing particles without calcium.

FIGS. 4 and 5 show the results of transfection of further cell lines with delivery systems having calcium- and silicon-containing particles.

FIG. 6 is a transmission electron microscope (TEM) image that shows the aggregation of silicon particles, as described herein.

### DETAILED DESCRIPTION

While the subject-matter of the present disclosure is described and illustrated below with reference to particular embodiments, it will be appreciated by those skilled in the art that the subject-matter lends itself to many different variations not specifically illustrated herein. By way of example only, certain possible variations of the present disclosure in all its aspects will now be described.

### Particles Comprising One or More Metals and Hydrolysable Silicon

As used herein, the term “metal” has its normal meaning in the chemical arts.

Metals are generally considered to be Li, Be, Na, Mg, Al, K, Ca, Sc, Ti, V, Cr, Mn, Fe, Co, Ni, Cu, Zn, Ga, Rb, Sr, Y, Zr, Nb, Mo, Tc, Ru, Rh, Pd, Ag, Cd, In, Sn, Cs, Ba, La, Ce, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, Fr, Ra, Ac, Th, Pa, U, Np, Pu, Am, Cm, Bk, Cf, Es, Fm, Md, No, Lr, Rf, Db, Sg, Bh and Hs. Meanwhile, B, Si, Ge, As, Sb and Te are generally considered to be metalloids. For the purposes of the present disclosure, B, Si, Ge, As, Sb and Te are taken to not be metals.

The one or more metals may be incorporated in (especially, dispersed throughout) a hydrolysable silicon matrix, (i) substitutionally, (ii) interstitially and/or (iii) via surface attachment. In this way, an improved API delivery vehicle may be provided. In particular, calcium (especially Ca²⁺ ions) may be dispersed substitutionally and/or interstitially throughout a hydrolysable silicon matrix.

The one or more metals may especially comprise or consist of one or more alkaline earth metals, i.e. one or more of Be, Mg, Ca, Sr, Ba and Ra. If so, they especially comprise or consist of one or both of magnesium and calcium (such as Ca²⁺ and/or Mg²⁺ ions), more especially calcium, especially calcium present as Ca²⁺.

Preferably, the one or more metals comprise or consist of one or more of lithium, sodium, potassium, magnesium and calcium; especially magnesium and calcium (such as Ca²⁺ and/or Mg²⁺ ions); most especially calcium, above all calcium present as Ca²⁺.

When the one or more metal is calcium, as is preferred, calcium may optionally be the only metal present (preferably, present as Ca²⁺). Alternatively, other metals may be present in addition, especially magnesium (preferably, in such cases, the majority metal is calcium, preferably, present as Ca²⁺; for example, calcium may be present at about 95% by weight of total metals; preferably, Ca²⁺ may be present at about 95% by weight of total metals).

Elemental metals are generally liable to form cations upon reaction and are generally liable to form basic oxides upon reaction with oxygen (or oxygen-containing species). It will be understood that the one or more metals of the particles disclosed herein may be present as metal atoms. The one or more metals may be present as metal ions, especially cations (metals typically form cations upon ionisation). The one or more metals may be present as a mixture of ions and atoms.

The one or more metals may be calcium present as Ca²⁺; Ca; or Ca²⁺ and Ca, dispersed in (preferably, throughout) a hydrolysable silicon matrix (preferably, including in the cores of the particles). Especially, the one or more metals may be calcium present as Ca²⁺ dispersed in (preferably, throughout) a hydrolysable silicon matrix (preferably, including in the cores of the particles).

The one or more metals may be magnesium present as Mg²⁺; Mg; or Mg²⁺ and Mg, dispersed in (preferably, throughout) a hydrolysable silicon matrix (preferably, including in the cores of the particles). Especially, the one or more metals may be magnesium present as Mg²⁺ dispersed in (preferably, throughout) a hydrolysable silicon matrix (preferably, including in the cores of the particles).

As will be appreciated from the foregoing disclosure, the particles comprising one or more metals and hydrolysable silicon may preferably be particles of hydrolysable silicon having one or both of metal atoms and metal ions distributed in (preferably, dispersed throughout) a hydrolysable silicon matrix. In particular, calcium (especially, Ca²⁺ ions) may be distributed in (preferably, dispersed throughout) a hydrolysable silicon matrix (preferably, including in the cores of the particles).

The metal atoms and metal ions (such as calcium, especially Ca²⁺) may thus be dispersed throughout the hydrolysable silicon matrix of the particles, including in the particles' cores. As used herein, the term “core” refers to a volume extending from the centre of a particle and optionally but not necessarily excluding the exterior surface of the particle.

The distribution may especially be substantially homogenous. In particular, Ca²⁺ and/or Mg²⁺ ions (especially, Ca²⁺ ions) may be distributed homogeneously throughout a hydrolysable silicon matrix.

The particles may optionally be characterised as comprising or consisting of a solid solution of one or both of metal atoms and metal ions in a hydrolysable silicon matrix, the hydrolysable silicon matrix being a “solvent” to the “solute” metal(s) (such as a solid solution of calcium, especially Ca²⁺, in a hydrolysable silicon matrix).

Atoms and/or ions of the one or more metals may be intercalated in the hydrolysable silicon matrix (such as calcium, especially Ca²⁺, intercalated in a hydrolysable silicon matrix). Additionally or alternatively, atoms and/or ions of the one or more metals may be substitutional (i.e., take the place of) silicon atoms in the hydrolysable silicon matrix (such as calcium, especially Ca²⁺, substituting silicon atoms in a hydrolysable silicon matrix).

It will be understood that preferably, the one or more metals are not present in the form of macro- or microscopic fragments of elemental metal. Nor, preferably, is there a coating of the one or more metals on the surface of the particles. Preferably, the particles are instead characterised by having metal atoms and/or metal ions distributed in (especially, dispersed throughout) a hydrolysable silicon matrix. Calcium, especially Ca²⁺ ions, may preferably be dispersed throughout a hydrolysable silicon matrix.

Preferably, the calcium (especially, Ca²⁺ ions) dispersed throughout the hydrolysable silicon matrix includes calcium (especially, Ca²⁺ ions) dispersed within the cores of the particles (i.e. infiltrating to the cores of the particles, as opposed to forming a coating on the surface of each particle without infiltrating to the core of each particle).

Metal atoms and/or ions (especially calcium, most especially Ca²⁺ ions) being distributed in a hydrolysable silicon matrix may encompass the metal atoms and/or ions (especially calcium, most especially Ca²⁺ ions) being dispersed in a hydrolysable silicon matrix. References herein to being “dispersed in” or “dispersed throughout” the matrix may mean being present throughout substantially the entire volume of the matrix. Metal atoms and/or ions (especially calcium, most especially Ca²⁺ ions) being dispersed in a hydrolysable silicon matrix may encompass there being a solid solution of the metal atoms and/or ions (especially calcium, most especially Ca²⁺ ions) in a hydrolysable silicon matrix, the metal atoms and/or ions (especially calcium, most especially Ca²⁺ ions) being intercalated in a hydrolysable silicon matrix, and/or the metal atoms and/or ions (especially calcium, most especially Ca²⁺ ions) substituting Si atoms in a hydrolysable silicon matrix.

Optionally, while there may not be a coating of the one or more metals on the surface of the particles, atoms and/or ions of the one or more metals may be concentrated on the surface of the particles. Thus, optionally, the particles may comprise or consist of particles having a core formed of a hydrolysable silicon matrix, surrounded by a shell formed of a hydrolysable silicon matrix wherein there are atoms and/or ions of the one or more metals. Additionally or alternatively, the core formed of a hydrolysable silicon matrix may have a surface to which atoms and/or ions of the one or more metals are bound. In other words, there may be particles of hydrolysable silicon having their surfaces “decorated” with atoms and/or ions of the one or more metals.

Alternatively, atoms and/or ions of the one or more metals may be concentrated in an outer shell of the particles. If so, the particles may have a core consisting substantially of silicon.

In certain preferred embodiments, the particles are formed of a hydrolysable silicon matrix, throughout which metal ions (especially Mg²⁺ or Ca²⁺ ions, most especially Ca²⁺ ions) are distributed.

Alternatively, in other preferred embodiments, the particles are formed of a hydrolysable silicon matrix, on or close to the surface of which metal ions (especially Mg²⁺ or Ca²⁺ ions, most especially Ca²⁺ ions) are distributed.

The particles are preferably biodegradable and non-toxic. In vivo, the particles preferably degrade completely to orthosilicic acid and other soluble, non-toxic species capable of being excreted by humans.

Preferably, the particles contain at least about 50% by weight silicon atoms relative to the total weight of the particles. They may contain at least about 60, about 70, about 80, about 90, especially about 95% by weight silicon atoms relative to the total weight of the particles.

In line with the present disclosure, the hydrolysable silicon may preferably be a major constituent of the particles, making up at least 70, especially at least 90, more especially at least 95% by weight of the particles.

The particles may optionally contain at least 600 ppm of the one or more metals, especially at least 700 ppm of the one or more metals, more especially at least 800 ppm of the one or more metals (especially when the one or more metals are or comprise calcium, most especially Ca²⁺).

The particles may optionally contain at most 11,000 ppm of the one or more metals, especially at most 10,000 ppm of the one or more metals, more especially at most 900 ppm of the one or more metals (especially when the one or more metals are or comprise calcium, most especially Ca²⁺).

Thus, the particles may contain at least 700 ppm of the one or more metals, and at most 10,000 ppm of the one or more metals (especially when the one or more metals are or comprise calcium, most especially Ca²⁺).

It will be understood that where ppm values are expressed for the one or more metals, the remainder of the particles may be or consist essentially of substantially pure silicon.

The hydrolysable silicon content of the particles may be substantially pure silicon. If not, it contains at least about 50% by weight silicon atoms. It may contain at least about 60, at least about 70, at least about 80, at least about 90, especially at least about 95% by weight silicon atoms.

Preferably, therefore, the particles comprising one or more metals and hydrolysable silicon contain at least about 50% by weight silicon atoms relative to the total weight of the particles comprising one or more metals and hydrolysable silicon. They may contain at least about 60, at least about 70, at least about 80, at least about 90, and especially at least about 95% by weight silicon atoms relative to the total weight of the particles comprising one or more metals and hydrolysable silicon.

A sample of the hydrolysable silicon may show a rate of hydrolysis, for example in PBS buffer at room temperature, of at least about 10% (preferably, at least about 50%) of the rate of hydrolysis of a sample of pure silicon of the same dimensions. Assays for hydrolysis of silicon-containing material are widely known in the art; see, for example, WO 2011/001456, incorporated herein by reference in its entirety.

Although the particles may contain traces of silica, silica is not hydrolysable silicon.

At least about half of the silicon atoms in the particles may be in the form of elemental silicon.

The particles may, especially, be nanoparticles. The nanoparticles may have a mean diameter in a range of about 1 nm to about 500 nm, especially about 1 nm to about 250 nm, more especially about 1 nm to about 100 nm, preferably about 1 nm to about 50 nm, especially about 3 nm to about 50 nm, more especially about 3 nm to about 30 nm, (such as about 10 nm).

A particularly preferred range for the nanoparticles' mean diameter is about 1 nm to about 30 nm. This may have advantages in terms of the nanoparticles' aggregation, as described hereinbelow.

The dimensions of nanoparticulate objects, including the particles' mean diameter, may be measured, for example, by scanning electron microscopy (SEM) or by transmission electron microscopy (TEM).

The particles may be porous, especially mesoporous. Particles comprising hydrolysable silicon can be made porous by standard techniques such as contacting the particles with a hydrofluoric acid (HF)/ethanol mixture and applying a current. By varying the HF concentration and the current density and time of exposure, the density of pores and their size can be controlled and can be monitored by scanning electron micrography and/or nitrogen adsorption desorption volumetric isothermic measurement. If the particles are porous, their total surface area will be increased by virtue of their porosity. For example, their surface area may be increased by at least about 50% or at least about 100%, compared to the surface area of a corresponding non-porous particle. In many circumstances, porous particles will in reality have a much greater increase in total surface area by virtue of their porosity. According to certain embodiments the porosity is at least about 30, about 40, about 50 or about 60%; meaning that, respectively, at least about 30, about 40, about 50 or about 60% of the particle volume is pore space.

Average pore diameter may be in a range of from about 0.1 nm to about 10 nm, for example from about 0.1 nm to about 3 nm, such as about 2 nm.

It will be appreciated that the particles may be produced by various techniques familiar to the skilled person.

The techniques may include, for example, purely physical (sometimes referred to, in the art, as “non-wet”) processes having bulk silicon (especially, silicon wafer) as the starting material; such as pulsed laser ablation, thermal degradation and ball milling. Thus, the particles may be obtainable by a method comprising or consisting of one or more of pulsed laser ablation, thermal degradation and ball milling, of bulk silicon (especially, silicon wafer).

Additionally or alternatively, the particles may be produced by chemical (sometimes referred to, in the art, as “wet”) techniques, including but not limited to electrochemical etching of bulk silicon (especially, silicon wafer). Such techniques optionally include the HF etching described above. Thus, the particles may be obtainable by a method comprising or consisting of electrochemical etching of bulk silicon (especially, silicon wafer).

Once formed, silicon particles may be sorted by size, such, for example, as by air classification, sieving and/or filtration. Thus, the particles may be obtainable by a method comprising one or more of air classification, sieving and/or filtration.

Thus, for example, the particles may be obtainable by a method comprising producing silicon particles from bulk silicon, especially from silicon wafer, such as by one or more of pulsed laser ablation, thermal degradation, ball milling, and electrochemical etching, of bulk silicon (especially, silicon wafer); followed, optionally, by sorting by size, such as by air classification, sieving and/or filtration.

Optionally, the particles may be washed before use, such as in methanol or ethanol, to remove a thin oxidised layer from their surface. In the art, this may be termed “activation”.

The particles thus obtained may have a narrow size distribution and homogeneous surface chemistry, leading to batch-to-batch reliability and reproducibility of one or more of the advantages described herein.

Suitable physical and chemical techniques are set out, for example, in WO 2011/012867 A1 (in the name of SISAF LTD); in Tokarska K et al., Facile production of ultra-fine silicon nanoparticles, R. Soc. Open Sci., 2020, 7: 200736; and in Kim, T., Lee, J. Silicon nanoparticles: fabrication, characterization, application and perspectives, Micro and Nano Syst. Lett, 2023, 11:18, each of which is incorporated herein by reference in its entirety.

Advantageously, adding one or more metals to hydrolysable silicon (especially, dispersing calcium, most especially Ca²⁺, in a hydrolysable silicon matrix) may have a beneficial effect on the zeta potential of the particles, which may be a proxy for the surface charge of the particles. Adding one or more metals may provide a zeta potential more suitable for improved loading of an API. It is thought that this may help stabilise an API while it is in circulation in vivo until it reaches a target cell, where it is subsequently released. Thus, more API may reach a target cell in a given time period after administration, compared to when particles without the one or more metals are used, leading to more efficient API delivery. When the one or more metals (especially calcium, most especially Ca²⁺) are dispersed in the hydrolysable silicon matrix of the particle, thus infiltrating to the core of the particle, it is thought that this effect may be maintained as the particles degrade in vivo, because substantially constant surface properties may be maintained as the particle shrinks. Whereas, a surface-coated particle would not have the same stable surface properties once the surface coating is lost during in vivo degradation.

Without wishing to be bound by theory, it is also thought that when the API is a nucleic acid, its phosphate backbone may interact with the one or more metals (especially calcium, most especially Ca²⁺) and the hydrolysable silicon in such a way as to provide additional control over the release of the nucleic acid API. This may offer the opportunity to use less or no amine-rich cationic lipid, compared to traditional liposomal delivery vehicles that rely on amine-rich cationic lipids, having a risk of toxicity, for their interaction with nucleic acid APIs.

Adding the one or more metals (especially calcium, most especially Ca²⁺) to hydrolysable silicon in the particles may increase the half-life in vivo (at about pH 7.4 and about 37° C.) of the API, such as by a factor of about 1.5, especially a factor of about 2. Especially, in the presence of the particles and the one or more lipids disclosed herein, the API may have a half-life in vivo of more than about 1 hour, especially more than about 2 hours, more especially more than about 6 hours. It will be appreciated that the term “half-life in vivo of the API”, as used herein, may refer to the elimination half-life in vivo of the API, i.e. the time period taken for the amount of the API, once administered, to reduce by about half. It will also be appreciated that the term “amount of the API” may refer to the amount of the API or a derivative thereof having the same or substantially the same intended pharmaceutical effect. It will also be appreciated that the term “half-life in vivo”, as used herein, may refer to a systemic half-life of the API (inside the body, especially the human body); as opposed to the half-life of the API on an external body surface, such as when present on the skin surface (outside the body, even if in contact with it).

The addition of the one or more metals (especially calcium, most especially Ca²⁺) to hydrolysable silicon in the particles may optionally change their zeta potential by a magnitude of at least about 5 mV, especially at least about 10 mV, compared to particles of hydrolysable silicon without the one or more metals (especially, without calcium, such as without Ca²⁺).

Adding the one or more metals (especially, Ca²⁺ and/or Mg²⁺, most especially Ca²⁺) may lead to a more positive zeta potential, leading to improved binding with an API having a net negative charge, especially an API having a net negative charge at a pH of about 7.4 (because this is a typical physiological pH), especially a nucleic acid, more especially linear and/or circular RNA, most especially mRNA, tRNA or siRNA. Thus, in some embodiments, the API is a negatively charged API, especially a nucleic acid, more especially mRNA, tRNA or siRNA.

Adding the one or more metals (especially, Ca²⁺ and/or Mg²⁺, most especially Ca²⁺) may still provide improved binding with an API having a net positive charge, especially an API having a net positive charge at a pH of about 7.4 (because this is a typical physiological pH), especially where the particles' zeta potential is further modulated by other components such as the one or more lipids (e.g., phospholipid(s)) being present.

The addition of the one or more metals (especially, Ca²⁺ and/or Mg²⁺, most especially Ca²⁺) to hydrolysable silicon in the particles may optionally increase their zeta potential by at least about 5 mV, especially at least about 10 mV, compared to particles of hydrolysable silicon without the one or more metals.

The addition of the one or more metals (especially, Ca²⁺ and/or Mg²⁺, most especially Ca²⁺) to hydrolysable silicon in the particles may lead to improved binding with zwitterionic or neutral APIs. Thus, in some embodiments, the API is zwitterionic or neutral.

Conventional liposomal transfection compositions (which do not comprise silicon) tend to comprise a significant quantity (such as a majority % by weight) of cationic lipid, especially amine-rich cationic lipid; wherein the positive charge of the cationic lipid is intended to stabilise negatively charged APIs, particularly nucleic acid. However, such cationic lipid may not be cost-effective and may potentially not have an adequate safety profile for all clinical applications, such as administration to certain patient groups, e.g. infants, the elderly, or pregnant women. As described herein, the particles provide the potential to use less or no cationic lipid, especially less or no “exotic” or amine-rich cationic lipid, thus may provide improved cost-effectiveness and safety.

Relatedly, the particles' zeta potential may also be modulated by other components of the composition.

Adding one or more metals (especially, Ca²⁺ and/or Mg²⁺, most especially Ca²⁺) to hydrolysable silicon in the particles may mean that components having certain beneficial functionalities can be added that would otherwise not be conducive to satisfactory API binding and delivery.

It will be understood that a change in zeta potential which may result from adding one or more metals to hydrolysable silicon, may not be the only beneficial effect of the addition of the one or more metals (such as calcium, especially Ca²⁺). A change in zeta potential may not be the only reason for increased success of API binding and delivery. Also, as described herein, other components besides the one or more metals (such as calcium, especially Ca²⁺) and the hydrolysable silicon may influence zeta potential and/or API binding and delivery.

Meanwhile, the particles and lipids in accordance with the present disclosure can be prepared, stored, and delivered to a clinic, then complexed with the API, such as mRNA, before being administered to a patient. The particles and lipids are able to be stored separately from the API, until close to the time when the API needs to be administered to a patient. Since the particles and lipids are not complexed with the reactive API such as mRNA while being stored, there is no need for storage at especially cold temperatures such as below 4° C. in order specifically to stabilise the API. This is a different approach to conventional lipid nanoparticle delivery vehicles, which are usually stored already encapsulating an API.

Moreover, the particles and lipids disclosed herein have a stabilising effect on a reactive API, such as a nucleic acid (especially, circular and/or linear RNA, most especially circular and/or linear mRNA) when complexed with it. As a result, the particles and lipids of the disclosure may optionally be stored already complexed to the API. This may be possible at not especially cold temperatures. For example, the composition comprising the API could be stored at temperatures of about 0° C. or above, especially of about 3-5° C., i.e. the temperature of a typical commercially available refrigerator; or at about 15-30° C., i.e. typical room temperature.

The particles and lipids can also stabilise APIs while they are circulating in the body. Additionally or alternatively, they can ensure efficient API uptake by cells. Additionally or alternatively, they can stabilise the API in the cytoplasm of a cell. For example, this may be advantageous where the API is mRNA which is preferably delivered safely to ribosomes in the cell cytoplasm for translation. Without wishing to be bound by theory, it is thought that the presence of Ca²⁺, in particular, may modulate cell and/or tissue targeting. Additionally or alternatively, the presence of Ca²⁺ may improve API uptake by cells, for example because of the role of Ca²⁺ in cellular signalling. Mg²⁺ may have an analogous effect.

### Lipids

The one or more lipids may be bound to the surface of the particles comprising one or more metals (especially, Ca²⁺ and/or Mg²⁺, most especially Ca²⁺) and hydrolysable silicon. The one or more lipids may be complexed with the API, such as a nucleic acid. The one or more lipids may comprise an ionisable lipid. The one or more lipids may comprise a lipid which has a net positive charge at a pH of about 7.4, also referred to herein as a “cationic lipid.”

It has been found that surface treating the particles comprising one or more metals (especially, Ca²⁺ and/or Mg²⁺, most especially Ca²⁺) and hydrolysable silicon, with one or more lipids may help to control the rate of release of the API (e.g. nucleic acid, especially mRNA or siRNA). The type of lipid used to treat the surface of the particles may help modulate the rate of API release.

Treating the particles comprising one or more metals (especially, Ca²⁺ and/or Mg²⁺, most especially Ca²⁺) and hydrolysable silicon with one or more lipids may have a beneficial effect on the surface charge of the particles. It may provide a zeta potential more suitable for improved loading of an API, especially a nucleic acid (such, for example, as siRNA, short activating RNA, short hairpin RNA or mRNA). It may help to control the rate of API release at a target site.

By way of example, treatment of the particles with phosphatidylcholine (PC), phosphatidylethanolamine (PE) and/or lecithin may promote a negative zeta potential (with a zeta potential ranging from about-60 to about-20 mV). Meanwhile, treatment with stearylamine and/or DOTAP may promote a positive zeta potential (with a zeta potential ranging from about 0 to about +40 mV). As described herein, adding one or more metals (especially, Ca²⁺ and/or Mg²⁺, most especially Ca²⁺) to a hydrolysable silicon matrix of the particles, modulates their zeta potential. Consequently, the particles when treated with a cationic lipid can more easily achieve a more positive zeta potential. For example, treatment of the particles with stearylamine and/or DOTAP can achieve a zeta potential of about +20 mV to about +60 mV. In consequence, a positive zeta potential can be achieved with a lower amount of cationic lipid or with a wider range of cationic lipids (including those which are non-toxic; such as those which are not amine-rich) compared to conventional liposomal API delivery vehicles. Even if the cationic lipid degrades during storage, resulting in a partial loss of positive charge of the lipid, the zeta potential of the particles can remain more comfortably positive for longer.

The one or more lipids preferably exclude toxic lipids. The one or more lipids may exclude exogenous lipids having a positive charge at physiological pH (known in the art as exotic cationic lipids); such lipids may have an unfavourable toxicity profile, for example by triggering an innate immune response upon administration to a human patient, especially when they are amine-rich. Although well-suited to the electrostatic loading of polyanionic nucleic acids (including various form of RNA) these (amine-rich) exotic lipid species may lead to cytotoxicity, immunogenicity, and may even lead, contrary to what is desired, to non-specific tissue accumulation, thus counteracting the aim of targeted delivery. Furthermore, they may be complex to synthesise and may therefore be expensive.

Thus, the one or more lipids may exclude amine-rich lipid. Amine-rich lipids may be defined as having more than 2, 3 or 4 nitrogen atoms per molecule of lipid. In sharp contrast, the one or more lipids preferably contain up to 1 nitrogen atom per molecule of lipid.

Moreover, it has been found that the beneficial effects disclosed herein may be achieved without necessarily being tied to one or more specific lipid compounds. The one or more lipids may play a role in charge-charge interactions, such as in modulating the zeta potential at the surface of silicon particles, so as to enable better binding of the API. This effect may be seen across a wide range of lipids. Lipids as a class show trends in properties, especially in terms of inter-molecular interactions, enabling the compositions of the present disclosure to be implemented with different lipids and in differing amounts, compared to the specific lipids disclosed in the Examples below.

The one or more lipids may help to modulate the rate of hydrolysis of the hydrolysable silicon, such that the silicon hydrolyses to bioavailable orthosilicic acid (OSA) degradation product; rather than insoluble polymeric hydrolysis products. Controlling the rate of hydrolysis of the silicon may influence the rate of release of API associated with the silicon. Controlling the rate of API release may modulate the length of the time period during which protection of the API is sustained, especially concerning protection in vivo in the presence of various bodily fluids. Thus, more API may be delivered to a target cell in a given time period, than for an otherwise identical composition.

Lipids are generally understood to include fatty acids and fatty acid derivatives, glycerolipids, glycerophospholipids, sphingolipids, saccharolipids and polyketides. As used in the present application, the term “lipid” may encompass lipidated oligopeptide (a term used interchangeably herein with the term lipopeptide) wherein a short peptide sequence (such as a peptide sequence having 3 to 20 amino acid residues, such as 5 to 15 amino acid residues, especially 3, 4, or 5 amino acid residues, and most especially 5 amino acid residues) is conjugated to one or more fatty acid chains (especially a fatty acid chain having a 10 to 24 carbon chain length, preferably, a 12 to 18 carbon chain length; for example a 14, 15 or 16 carbon chain length; for instance, the peptide moiety may optionally be lipidated with a palmitoyl, cetyl or myristoyl moiety).

Without wishing to be bound by theory, while some lipids are investigated in the Examples hereinbelow, the mechanism by which the one or more lipids act may be due to properties of the class of lipids, such as their behaviour in response to charge-charge interactions; and may thus be generalisable beyond the exemplified lipids.

The one or more lipids may thus comprise one or more lipidated oligopeptides. Preferably, the one or more lipidated oligopeptides each comprise a fatty acid chain having in the range of about 12 to about 18 carbon atoms.

Preferably, the one or more lipidated oligopeptides each comprise 3 to 20 amino acid residues. Thus, the lipidated oligopeptide may be a lipdated tetrapeptide, lipidated pentapeptide or lipidated hexapeptide.

Preferably, the amino acid residues include at least one amino acid residue (for example, about 2 or about 3 amino acid residues) that is cationic at a pH of about 7.4 (physiological pH), such, for example, as lysine or arginine. For example, the lipidated oligopeptide may include one or more (for example, about 2) lysine resides.

An especial example of a lipidated oligopeptide (“lipopeptide”) is palmitoyl-pentapeptide-4 (CAS number 214047-00-4; abbreviated as PAL-KTTKS).

Thus, preferably, the one or more lipids may comprise or be one or more lipidated oligopeptides, particularly those having one or more amino acid residues that is or are positively charged at a pH of about 7.4 (i.e., about physiological pH) such, for example, as one or both of lysine and arginine.

The lipidated oligopeptide may especially be used in combination with one or more phospholipids, such as DOPE or DPPC. The alkyl chain of a lipidated oligopeptide molecule may be assimilated in a phospholipid bilayer, while the surface of the bilayer is decorated with the peptide moiety. Without wishing to be bound by theory, it is thought that a peptide moiety of the lipidated oligopeptide can enable the targeting of one or more specific tissues and/or cells. Meanwhile, where the peptide moiety bears a positive charge at a pH of about 7.4 (i.e., about physiological pH), it may stabilise negatively charged APIs (e.g. nucleic acids, especially mRNA or siRNA).

The one or more lipids may be or comprise one or more of: one or more cationic lipids (e.g. DOTAP); one or more phospholipids (e.g. DOPE); and one or more polyethylene glycol (PEG) lipids (e.g. DSPE-PEG₂₀₀₀).

The one or more lipids may be or comprise one or more structural lipids (e.g. a cholesterol-based lipid). However, the one or more lipids may optionally exclude structural lipid. Thus, the one or more lipids may exclude sterols; especially, they may exclude cholesterol. It has been found that the presently disclosed compositions need not rely on these types of lipid, which traditional API delivery systems typically rely on. Thus, the compositions disclosed herein have the potential to provide alternatives to API delivery systems reliant on these types of lipids, especially cholesterol. Where cholesterol is not available or where its use is otherwise not possible (e.g., due to its effect in the body) this may be advantageous.

The one or more lipids may optionally include one or more of: phosphatidylcholine (PC); hydrogenated PC; stearylamine (SA); dioleoylphosphatidylethanolamine (DOPE); cholesteryl 3β-N-(dimethylaminoethyl) carbamate hydrochloride (DC-chol); 1,2-dioleoyl-3-trimethylammonium-propane (DOTAP); PEGylated 1,2-Distearoyl-sn-glycero-3-phosphoethanolamine (DSPE), such as DSPE-PEG₂₀₀₀; and derivatives thereof.

In certain embodiments, the lipid is selected from the group consisting of phosphatidylethanolamine (PE), phosphatidylcholine (PC), stearylamine (SA), or any combination thereof.

The lipid or lipids component may, in some embodiments, be or comprise a cationic lipid. The term “cationic lipid” refers to molecules having a net positive charge at pH 7.4 (physiological pH), having a cationic head group attached via some spacer to a hydrophobic tail. Examples include DTDTMA (ditetradecyl trimethyl ammonium), DOTMA (2,3-dioleyloxypropyl-1-trimentyl ammonium), DHDTMA (dihexadecyl trimethyl ammonium); dioleoyl-3-trimethylammonium propane (DOTAP); and stearylamine (SA). The positive charge may typically be stabilised by a negative counterion.

Thus, the one or more lipids may optionally be or comprise DOTAP. DOTAP exists in an S and an R enantiomeric form, and may be present as the S-, R-form or as a racemate. Optionally, of the total DOTAP present by weight, the R and S forms may be in approximately equal amounts (i.e. no more than about 60% of the total DOTAP present by weight, of either form). In other embodiments at least about 80, 90, 95, 98, or 99% of total DOTAP is in the R-form. In other embodiments at least about 80, 90, 95, 98, or 99% of total DOTAP is in the S-form.

Nonetheless, as described herein, addition of one or more metals (especially, Ca²⁺ and/or Mg²⁺, most especially Ca²⁺) to hydrolysable silicon in the particles of the present disclosure may enable less cationic lipid, especially less toxic or “exotic” cationic lipid, to be used, compared to conventional compositions for API delivery (such as lipid nanoparticles which comprise cationic lipid). While exotic cationic lipids may be suitable for electrostatic loading of polyanionic nucleic acids (including various form of RNA) their amine-rich nature may cause cytotoxicity, immunogenicity, and non-specific tissue accumulation.

Thus, the one or more lipids may optionally exclude cationic lipid, especially toxic cationic lipid. As described herein, cationic lipid may not be necessary.

Thus, the one or more lipids may be or comprise one or more of: one or more phospholipids (e.g. DOPE); and one or more polyethylene glycol (PEG) lipids (e.g. DSPE-PEG₂₀₀₀).

Overall, the particles disclosed herein can provide the potential to use less lipid (especially less cationic lipid, most especially less of a toxic cationic lipid) in API delivery vehicles, compared to conventional API delivery vehicles which do not contain the particles (e.g. conventional liposomal nucleic acid delivery vehicles, such as those typically used for mRNA delivery in vivo). Additionally or alternatively, the particles can provide the potential for API delivery vehicles to be formulated with a wider range of lipids while still providing transfection efficiency, storage stability, and/or targeted delivery to a particular type of tissue, or to a particular type of cell. In turn, this may lead to reduced reliance in the field on specific lipids, particularly cationic lipids, especially toxic cationic lipids and/or cationic lipids which are formulated specifically for the purpose of API delivery and which may therefore not be cost-effective or easily accessible.

The one or more lipids may have an average molecular weight in the range of about 500 to about 1000.

The ratio by weight of the one or more lipids (by which is meant all lipid components in the composition) to the particles may be in a range of from about 40:1 to about 1:1, especially a range of about 20:1 to about 1:1; such, for example, as a ratio of about 16:1, when the components are assembled for manufacture of a delivery system, i.e. before any further processing is carried out (such further processing may be, for example, the step “Extrusion” of “Materials and Methods” of Example 1 hereinbelow).

As described herein, the one or more lipids may especially comprise or be a phospholipid. The term “phospholipid”, as used herein, may refer to a lipid comprising a fatty acid chain and a phosphate group. Phospholipids may carry a negative charge, unlike a cationic lipid which is positively charged. However, phospholipids are typically zwitterionic compounds comprising both positive and negatively charged components, resulting in no overall charge. As such, phospholipids are typically classified as neutral lipids.

Suitable phospholipids may be or include glycerophospholipids. Especially suitable phospholipids may be or include those in which the polar head group is linked to quaternary ammonium moieties, such as phosphatidylcholine (PC) or hydrogenated phosphatidylcholine. The phospholipid may be, or be derived from, lecithin. A preferred phospholipid is DOPE (phosphatidyl ethanolamine or 1,2-dioleoyl-sn-glycero-3-phosphoethanolamine).

Preferably, the side chain(s) of the phospholipid may be aliphatic side chain(s) with about 15 or more carbon atoms, or an ether side chain with about 6 or more repeating ether units, such as a polyethylene glycol or polypropylene glycol chain.

Lipids with ether side chains may be referred to as “PEG-lipids” or “PEG-ylated” lipids. Thus, as used in the present application, the term “lipid” may cover PEG lipids. Thus, according to preferred embodiments, the one or more lipids may comprise or be one or more polyethylene glycol (PEG) lipids, especially PEGylated DSPE, such as DSPE-PEG₂₀₀₀.

The one or more lipids may optionally comprise or consist substantially of phosphatidylcholine (PC), hydrogenated phosphatidylcholine, stearylamine (SA), or combinations thereof.

The one or more lipids may optionally comprise at least about 5% (e.g., at least about 30% or at least about 50%) by weight of PC, based on the total weight of the one or more lipids.

The one or more lipids may optionally comprise at least about 5% (e.g., at least about 30% or at least about 50%) by weight of hydrogenated PC, based on the total weight of the one or more lipids.

The one or more lipids may optionally comprise at least about 5% (e.g., at least about 30% or at least about 50%) by weight of SA, based on the total weight of the one or more lipids.

The one or more lipids may optionally comprise or consist essentially of PC and SA, optionally in a ratio by weight of PC to SA in a range of from about 1:1 to about 20:1.

The one or more lipids may optionally comprise or consist essentially of the combination of DOPE, SA, and DC-cholesterol (DC-chol). The ratio by weight of DOPE:SA may be in a range of from about 1:1 to about 10:1. The ratio by weight of DOPE:DC-cholesterol may be in a range of from about 1:1 to about 5:1. The ratio by weight of SA:DC-cholesterol may be in a range of from about 1:1 to about 1:5.

In certain preferred embodiments, the one or more lipids may optionally comprise or consist essentially of a combination of DOTAP, DOPE and a PEG-lipid (especially DSPE-PEG₂₀₀₀). The ratio by weight of DOTAP:DOPE may be in a range of from about 1:2 to about 2:1; such, for example, as about 1:1. The ratio by weight of DOTAP:PEG-lipid may be in a range of from about 10:1 to about 5:1; such, for example, as about 7:1. The ratio by weight of DOPE:PEG-lipid may be in a range of from about 10:1 to about 5:1; such, for example, as about 7:1.

Amino acid(s) The composition disclosed herein may comprise one or more amino acids. In its broadest sense, the term “amino acid” encompasses any artificial or naturally occurring organic compound containing an amine (—NH2) and carboxyl (—COOH) functional group. It includes α, β, γ and δ amino acids. It includes an amino acid in any chiral configuration. The amino acid may, especially, be a naturally occurring a amino acid. It may be a proteinogenic amino acid or a non-proteinogenic amino acid (such as carnitine, levothyroxine, hydroxyproline, ornithine or citrulline).

The one or more amino acids may help stabilise the particles containing one or more metals (such as Ca²⁺ and/or Mg²⁺, especially Ca²⁺) and hydrolysable silicon. In vivo, the one or more amino acids may help to modulate the rate of hydrolysis of the silicon, such that the silicon hydrolyses to bioavailable orthosilicic acid (OSA) degradation product; rather than insoluble polymeric hydrolysis products. In this way, the one or more amino acids may complement the function of the one or more lipids of the present disclosure. Controlling the rate of hydrolysis of the silicon may influence the rate of release of API associated with the silicon. Controlling the rate of API release may modulate the length of the time period during which protection of the API is sustained, especially concerning protection in vivo in the presence of various bodily fluids. Thus, more API may be delivered to a target cell in a given time period, than for an otherwise identical composition.

Without wishing to be bound by theory, while some amino acids are investigated in the Examples hereinbelow, the mechanism by which the one or more amino acids act may be due to properties of the class of amino acids, such as their behaviour in response to charge-charge interactions; and may thus be generalisable beyond the exemplified amino acids.

In preferred embodiments, the amino acid(s) may comprise or consist essentially of glycine.

Additionally or alternatively, amino acids which are neutral or positively charged at physiological pH (about pH 7.4), such as tyrosine or arginine, may stabilise negatively charged APIs (e.g. nucleic acids such as mRNA). Meanwhile, amino acids which are neutral or negatively charged at physiological pH (about pH 7.4) may stabilise positively charged APIs. Nonetheless, the interplay of charge-based and/or other interactions (for example steric interactions) resulting from the combination of the particles containing one or more metals (such as Ca²⁺ and/or Mg²⁺, especially Ca²⁺) and hydrolysable silicon, lipid(s) and amino acid(s) may be such that amino acid(s) which are positively charged at physiological pH may help to stabilise positively charged APIs, or amino acid(s) which are negatively charged at physiological pH may help to stabilise negatively charged APIs.

The ratio by weight of the one or more lipids (i.e. total lipid components) to the amino acid(s) may be in a range of from about 40:1 to about 1:1; such, for example, as about 32:1.

Optionally, the composition may specifically comprise the amino acid tyrosine in addition to the amino acid(s) described hereinabove. Optionally, the composition may specifically comprise tyrosine instead of the amino acid(s) described hereinabove. Thus, it will be understood that while tyrosine is an amino acid, it may optionally be present as a separate, further component, for the purposes of the present disclosure, distinct from the amino acid(s) described hereinabove. Thus, when tyrosine is present as a further component distinct from and in addition to the amino acid(s) described hereinabove it will be understood that the calculation of a ratio by weight of the one or more lipids to amino acid(s) in a range of from about 40:1 to about 1:1; such, for example, as about 32:1, disclosed above, does not include the amount of additional, distinct tyrosine.

### Non-Reducing Disaccharide

Additionally or alternatively, the composition may comprise one or more non-reducing disaccharides, especially trehalose. The ratio by weight of the one or more lipids (i.e. total lipid components) to non-reducing disaccharide may be in a range of from about 20:1 to about 1:1; such, for example, as about 16:1.

### APIs

It will be understood that the composition of the present disclosure may be described as a pharmaceutical composition, being for the delivery of an active pharmaceutical ingredient (API).

The API may, for example, be a fragile API. As used herein, the terms “fragile APIs” and “reactive APIs” may be interchangeable and may refer to APIs which (i) have a half-life upon storage in aqueous solution at about 25° C. of at most one week, measurable by NMR or by GC-MS; and/or (ii) have a half-life in vivo of under about an hour, measurable by assay of a biological sample.

The API may be any pharmaceutically active compound; thus, for example, it will be understood that the term “API” encompasses pro-drugs. Especially, the API may be a nucleic acid, more especially siRNA or mRNA. Meanwhile, in other preferred embodiments, the API may be a protein.

The API may be for administration by injection, orally, intranasally or topically; especially, by injection or orally.

### Nucleic Acid APIs

The API may preferably be or comprise a nucleic acid, especially RNA. The RNA may be small interfering RNA (siRNA), small activating RNA (saRNA), small hairpin RNA (shRNA), or messenger RNA (mRNA), especially mRNA (e.g., mRNA that encodes a protein of a pathogenic organism).

Other nucleic acids for use in accordance with the present disclosure include: double- and single-stranded DNA; DNA: RNA hybrids; peptide: DNA hybrids; and peptide: RNA hybrids.

RNA and DNA may be naturally occurring or chemically modified to enhance their therapeutic properties, such as enhanced activity, increased serum stability, reduced off-targeting and lower immunological activation. Chemical modifications to RNA and DNA may include any modifications commonly known in the art. As used herein, the term “naturally occurring” means of natural human or animal origin. It will be understood that a molecular structure that is the same as a naturally occurring molecular structure may nevertheless be synthesized in vitro, such as when mRNA is synthesized by in vitro transcription (IVT).

Thus, as used herein, the terms nucleic acid, DNA and RNA also include known types of modifications, for example, labels which are known in the art, methylation, “caps”, substitution of one or more of the naturally occurring nucleotides with an analogue, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

Similarly, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides will also include modifications on the sugar moiety, e.g., wherein one or more of the hydroxyl groups are replaced with a halogen, an aliphatic group, or are functionalized as ethers, amines, or the like. Other modifications to nucleotides or polynucleotides involve rearranging, appending, substituting for, or otherwise altering functional groups on the purine or pyrimidine base which form hydrogen bonds to a respective complementary pyrimidine or purine, e.g., isoguanine, isocysteine, and the like. In some embodiments, the oligonucleotides and/or probes include at least one, two, three or four modified nucleotides.

In some embodiments, the nucleic acids such as the RNAs disclosed herein include one or more universal bases. As used herein, the term “universal base” refers to a nucleotide analogue that can hybridize to more than one nucleotide selected from A, U/T, C, and G. In some embodiments, the universal base can be selected from the group consisting of deoxyinosine, 3-nitropyrrole, 4-nitroindole, 6-nitroindole, 5-nitroindole.

In its broadest sense, the term “saRNA” encompasses small activating RNA, comprising RNA molecules which operate within the RNA activation (RNAa) pathway. The saRNA may be double-stranded. The saRNA may have a length in a range of about 5 to about 50 base pairs, especially about 10 to about 40 base pairs, more especially about 10 to about 30 base pairs.

In its broadest sense, the term “shRNA” encompasses small hairpin RNA, comprising RNA molecules which operate within the RNA interference (RNAi) pathway. The shRNA may be single stranded while also having base pairing thereby forming a hairpin loop. The single strand of the shRNA may have a length in a range of about 10 to about 100 bases, especially about 25 to about 75 base pairs, more especially about 40 to about 70 base pairs; which may then form a hairpin loop.

In its broadest sense, the term “siRNA” encompasses small interfering RNA, comprising RNA molecules which operate within the RNA interference (RNAi) pathway. siRNA is sometimes known as short interfering RNA or silencing RNA. The siRNA may be double stranded. The siRNA may have a length in a range of from about 5 to about 50 base pairs, especially about 10 to about 40 base pairs, more especially about 15 to about 30 base pairs. Examples 4, 6 and 7 hereinbelow investigate siRNA APIs in the compositions of the present disclosure.

In its broadest sense, the term “tRNA” encompasses transfer RNA, comprising RNA molecules which serve, during protein synthesis, as links (or adaptors) between mRNA molecules and growing chains of amino acids. The primary structure of the tRNA may have a length in a range of from about 20 to about 200 nucleotides, especially about 50 to about 100 nucleotides. The tRNA may have a cloverleaf secondary structure. The tRNA may have an L-shaped tertiary structure.

In its broadest sense, the term “mRNA” encompasses messenger RNA for the synthesis of protein(s). It may encompass mRNA comprising a 5-prime cap and/or a poly-adenylated terminus. Alternatively, one or both of those features may be absent. Typically, the mRNA may be single stranded. The coding region of the mRNA may be at least about 100, especially at least about 500, more especially at least about 1000 bases in length.

The mRNA may encode an antigen, thereby providing a composition which is a vaccine. The antigen may be a bacterial, parasitic or fungal antigen. The antigen may be a viral antigen, especially a viral antigen of one of the viral diseases described hereinbelow; more especially an antigen of a respiratory virus, for example an antigen of SARS-COV-2, for example an antigen deriving from the spike protein of SARS-CoV-2.

The mRNA may encode multiple proteins, thereby providing more effective pharmacological activity. The mRNA may encode multiple antigens, especially multiple viral antigens.

The mRNA may additionally encode an adjuvanting protein. An adjuvant may additionally or alternatively be provided as a further component of the composition in addition to the API.

The mRNA may encode an allergen (including but not limited to one or more nut allergens; which in turn include, but are not limited to: one or more seed storage proteins, such as vicilins, legumins, albumins; one or more plant defence related proteins; and one or more profilins).

The mRNA may encode a protein that modulates an immune, autoimmune, or inflammatory disease (including, but not limited to, lupus, atherosclerosis, chronic obstructive pulmonary disease, inflammatory bowel disease, multiple sclerosis, psoriasis, a rheumatic disease, uveitis, atopic dermatitis, and pulmonary fibrosis).

The mRNA may encode a tumour-specific antigen. As used herein, the term tumour-specific antigen may refer to an antigen that arises, in one or more malignant cancer cells, from non-synonymous somatic mutation (leading to a neoantigen) or viral-integrated mutation (leading to an onco-viral antigen). Tumour-specific antigens may thus refer to antigens that are completely absent from (not expressed by) non-cancerous (healthy, normal) cells.

The mRNA may encode a tumour-associated antigen. As used herein, the term tumour-associated antigen may refer to an antigen that is over-expressed in a malignant cancer cell, compared to a non-cancerous (healthy, normal) cell, for example due to genetic amplification or post-translational modifications. The term tumour-associated antigen may encompass overexpressed antigens (which term may refer to proteins that are moderately expressed in non-cancerous (healthy, normal) cells, but expressed abundantly in malignant cancer cells); differentiation antigens (which term may refer to proteins that are selectively expressed by the cell lineage from which the malignant cells evolved, an example being prostate-specific antigen); and cancer-germline antigens (which term may refer to antigens that are normally limited to reproductive tissues, but which are aberrantly expressed in a malignant cancer cell; for example, melanoma antigen family A3 (MAGE-A3); New York Esophageal Squamous Cell Carcinoma-1 Antigen (NY-ESO-1); and Preferentially Expressed Antigen in Melanoma (PRAME)).

It is thought that the particles containing one or more metals (such as Ca²⁺ and/or Mg²⁺, especially Ca²⁺) and hydrolysable silicon can help the disclosed compositions deliver an API more effectively to a cell, especially where the API is a nucleic acid, more especially siRNA or mRNA. In particular, the compositions disclosed herein may mitigate or address the problem of how to ensure APIs reach cells once they have been administered to a patient, including how to stabilise APIs while they are circulating in the body. They may mitigate or meet the need for tissue or cell targeting, so that an API can be delivered to the correct cells. Once a target cell is reached, they may help ensure efficient API uptake by cells. Following uptake by a cell, they may help to ensure API stability in the cytoplasm; for example, stabilisation and release of an mRNA API in the cytoplasm over an appropriate time period so that more mRNA can be translated successfully. Without wishing to be bound by theory, it is thought that the presence of Ca²⁺, in particular, may modulate cell and/or tissue targeting. Additionally or alternatively, the presence of Ca²⁺ may improve API uptake by cells, for example because of the role of Ca²⁺ in cellular signalling. Analogous behaviour may be shown with Mg²⁺.

Accordingly, the method disclosed herein may comprise an in vivo step of transfecting a human cell with a nucleic acid.

The compositions disclosed herein for delivery of an API may be non-toxic. For example, they may be substantially fully biodegradable as described herein, especially in that the silicon may degrade to non-toxic orthosilicic acid in vivo. This may especially be the case when the one or more metals is/are or comprise Ca²⁺ and/or Mg²⁺, which are typically non-toxic.

The compositions disclosed herein for delivery of an API may be fully dispersible in an aqueous environment, to ensure ease of delivery, such as by injection in aqueous solution. This may especially be the case when the one or more metals is/are or comprise Ca²⁺ and/or Mg²⁺.

### Complexation of Components, Especially Particle/Lipid/API Complexation

Preferably, the particles, comprising one or more metals (such as Ca²⁺ and/or Mg²⁺, especially Ca²⁺) and hydrolysable silicon, are complexed with the one or more lipids thus forming a delivery vehicle for transport of the API. Thus, when the API is added, it also becomes complexed with the particles and/or the lipid. Thus, the composition contains a complex of the particles with the lipids and the API. Put another way, the particles and lipid are organised into a delivery vehicle that is loaded with the API. Advantageously, this may make the API less liable to react with one or more external reactive species. The API may be less at risk of degradation catalysed by enzymes external to the complex, especially in vivo, such as during circulation in the body and/or in a cell cytoplasm; this may especially be the case where the API is a nucleic acid, more especially mRNA.

In its broadest sense, as used here, the term “complexed with” may encompass ionic and/or covalent and/or physical interactions, and especially may encompass charge-charge interactions, such as those resulting from the particles' zeta potential.

Thus, preferably, the zeta potential of the particles, especially when modulated by the one or more lipids and any other components present, is such as to attract and facilitate binding of the API. This may especially be the case when the one or more metals is/are or comprise Ca²⁺ and/or Mg²⁺, most especially when the one or more metals is/are or comprise Ca²⁺.

In preferred embodiments wherein amino acid(s) is or are present, the amino acid(s) may also complex with the particles, lipid(s) and/or API. The amino acid(s), especially when charged, may modulate the particles' zeta potential thus modulating API and/or lipid complexation with the particles.

When other advantageous components are present, such as those described hereinbelow, they may also complex with the particles, lipid(s), amino acid(s) and/or API.

The one or more lipids may be formed of, or may comprise, one or more lipid structures. Said structures may be or comprise one or more of: micelles, incomplete micelles, liposomes, incomplete liposomes and (e.g., solid or semi-solid) lipid globules, as described herein.

Meanwhile, preferably, the API (especially, RNA, most especially mRNA) is bound (especially, bound non-covalently) to the particles comprising one or more metals (especially, calcium; most especially, Ca²⁺) and hydrolysable silicon (“the particles”). Preferably, at least about 50, 60 or 70% of API in the composition is bound to the particles in this way.

In turn, preferably, the particles are bound to the surface of, and/or are present in the interior of, the one or more lipid structures.

Particle-API binding may be especially effective at reducing or preventing API degradation if the particles aggregate into chains. Thus, preferably, the particles are aggregated, especially into chains. The presence of the one or more metals (especially, calcium; most especially, Ca²⁺) may facilitate such aggregation, for example by reducing particle-particle repulsion.

The chains may extent into the interior of lipidic structures (such as liposomes, incomplete liposomes, micelles, incomplete micelles and/or lipid globules) formed by the one or more lipids. API bound to particles in chains embedded in the interior of lipidic structures may be especially shielded from water molecules, thus shielded from degradation by hydrolysis.

The lipidic structures (such as liposomes, incomplete liposomes, micelles, incomplete micelles and/or lipid globules) may typically have a mean diameter of about 50 nm to about 500 nm; especially about 100 nm to about 500 nm. Meanwhile, the particles comprising one or more metals (especially, calcium; most especially, Ca²⁺) and hydrolysable silicon may typically have a mean diameter in a range of from about 1 nm to about 50 nm, such as about 1 nm to about 30 nm. This relative difference in size may optimise the formation of aggregates (especially, chains) of the particles being embedded in, and/or decorating the surface of, the lipidic structures.

Thus, preferably, particles are bound to the surface of, and/or are present in the interior of, one or more of: micelles, incomplete micelles, liposomes, incomplete liposomes, and (e.g., solid or semi-solid) lipid globules; and API is bound to the particles.

Preferably, particles are present in one or more aggregates of the particles, especially one or more aggregates comprising or consisting of chains of the particles, most especially chains that extend into the interior of the one or more lipid structures, such as into the interior of one or more of: micelles, incomplete micelles, liposomes, incomplete liposomes, and (e.g., solid or semi-solid) lipid globules (especially, liposomes and/or lipid globules). An amino acid (especially, glycine, arginine and/or tyrosine, such as glycine) may also be associated with (e.g., bound non-covalently to) the particles; and/or associated with (e.g., on the surface of) the one or more lipid structures.

As used herein, the term liposomal lipid particle, or the term liposome, may have its normal meaning in the art. Thus, it may refer to a vesicle having at least one lipid bilayer, which may be approximately spherical in shape. A liposome may be visualised as a lipid “bubble” surrounding an interior space. The interior space may be a hydrophilic environment.

The composition may in some embodiments comprise one or more liposomes. I.e., the one or more lipids may be formed of, or may comprise, one or more liposomes.

The composition may in some embodiments comprise incomplete liposomes. In that sense, their interior space may be accessible from the exterior. An incomplete liposome may be visualised as an incomplete lipid “bubble” wherein there are one or more gaps in the (approximately spherical) lipid bilayer surface. Thus, suitably, the one or more lipids may be formed of, or may comprise, one or more incomplete liposomes.

Suitably, the one or more lipids may be formed of, or may comprise, one or more incomplete liposomes and/or one or more (complete) liposomes.

Thus, the present composition may comprise the particles associated with one or more liposomes and/or one or more incomplete liposomes, wherein the API is associated with (especially, bound to) the particles. An amino acid (especially, glycine, arginine and/or tyrosine, such as glycine) may also be associated with the particles.

Optionally, the API may be encapsulated by the one or more lipids.

Liposomes are especially suitable for encapsulating APIs. Thus, the API may optionally be encapsulated in a liposome, or, preferably, the API may be partially encapsulated in an incomplete liposome. Where the liposome is an incomplete liposome, there exists a route for the API to cross between the liposome interior and exterior.

The optionally present liposomes or incomplete liposomes may have a mean diameter in a range of from about 50 nm to about 500 nm, especially about 50 nm to about 300 nm, more especially about 100 nm to about 300 nm.

The API may be associated non-covalently (especially, by virtue of ionic interactions) with the exterior surface of a liposome or of an incomplete liposome. The API may be located within the interior space of the (optionally, incomplete) liposome (i.e., at least partially encapsulated). Preferably, at least about 10% of the API is located within the interior space. Additionally or alternatively, at least 10% of the API may preferably be located in non-covalent association with the exterior surface.

Thus, at least about 10%, about 30%, or about 50% of the API may be fully encapsulated within the interior space of the (optionally, incomplete) liposome, with the remainder being located in non-covalent association with the exterior surface of the (optionally, incomplete) liposome.

API may be bound non-covalently to particles comprising one or more metals (especially, calcium; most especially, Ca²⁺) and hydrolysable silicon that are bound to the surface of one or more liposomes and/or one or more incomplete liposomes. Up to about 10 or 20% of the API may be bound non-covalently to particles that are, in turn, bound to the surface of one or more liposomes and/or one or more incomplete liposomes.

API may be bound non-covalently to particles comprising one or more metals (especially, calcium; most especially, Ca²⁺) and hydrolysable silicon that are, in turn, in the interior of one or more liposomes and/or one or more incomplete liposomes. At least about 50, 60 or 70% of the API may preferably be bound non-covalently to particles that are in the interior of one or more liposomes and/or one or more incomplete liposomes.

Preferably, API (especially, RNA, most especially mRNA) is bound non-covalently to particles comprising one or more metals (especially, calcium; most especially, Ca²⁺) and hydrolysable silicon that are in the interior of one or more liposomes and/or one or more incomplete liposomes; and API (especially, RNA, most especially mRNA) is bound non-covalently to particles that are bound to the surface of one or more liposomes and/or one or more incomplete liposomes.

In some embodiments, the composition may be free or substantially free of liposomes; and/or may be free or substantially free of incomplete liposomes.

Optionally, the one or more lipids may be formed of, or may comprise, one or more lipid monolayers. Optionally, the one or more lipids may be or comprise one or more micelles or incomplete micelles. It will be understood that micelles have similar characteristics to liposomes, except that micelles' walls are formed of lipid monolayer; whereas liposomes' walls are formed of lipid bilayer. Thus, a micelle may refer to a vesicle having at least one lipid monolayer, which may be approximately spherical in shape. A micelle, similarly to a liposome, may be visualised as a lipid “bubble” surrounding an interior space. The interior space may be a hydrophilic environment.

Thus, the present composition may comprise the particles (comprising one or more metals-especially, calcium; most especially, Ca²⁺— and hydrolysable silicon) associated with one or more micelles and/or one or more incomplete micelles, wherein the API is associated with (especially, bound to) the particles.

The optionally present micelles may have a mean diameter in a range of from about 50 nm to about 500 nm, especially about 50 nm to about 300 nm, more especially about 100 nm to about 300 nm.

API may be bound non-covalently to particles comprising one or more metals-especially, calcium; most especially, Ca²⁺— and hydrolysable silicon, the particles being bound to the surface of one or more micelles and/or one or more incomplete micelles. Up to about 10 or 20% of the API may be bound non-covalently to particles that are, in turn, bound to the surface of one or more micelles and/or one or more incomplete micelles.

API may be bound non-covalently to particles comprising one or more metals (especially, calcium; most especially, Ca²⁺) and hydrolysable silicon that are, in turn, in the interior of one or more micelles and/or one or more incomplete micelles. At least about 50, 60 or 70% of the API may preferably be bound non-covalently to particles that are in the interior of one or more micelles and/or one or more incomplete micelles.

Preferably, API (especially, RNA, most especially mRNA) is bound non-covalently to particles comprising one or more metals (especially, calcium; most especially, Ca²⁺) and hydrolysable silicon that are in the interior of one or more micelles and/or one or more incomplete micelles; and API (especially, RNA, most especially mRNA) is bound non-covalently to particles that are bound to the surface of one or more micelles and/or one or more incomplete micelles.

In some embodiments, the composition may be free or substantially free of micelles; and/or may be free or substantially free of incomplete micelles.

The one or more lipids may be formed of, or may comprise, one or more lipid globules, each globule optionally being surrounded by a layer of surfactants. Lipid globules do not enclose an interior space or cavity. Instead, they are solidly formed, or substantially solidly formed, of lipid, in which other components, such as the particles (comprising one or more metals-especially, calcium; most especially, Ca²⁺— and hydrolysable silicon) to which are bound API molecules, may be dispersed. Thus, the globules' interior may be studded with the particles comprising one or more metals (especially, calcium; most especially, Ca²⁺) and hydrolysable silicon; with API molecules, in turn, being bound (non-covalently) to the particles. Additionally or alternatively (preferably, additionally), the particles comprising one or more metals (especially, calcium; most especially, Ca²⁺) and hydrolysable silicon, to which are (non-covalently) bound API molecules, may be bound to the surface of one or more lipid globules.

Thus, preferably, the present composition comprises the particles comprising one or more metals (especially, calcium; most especially, Ca²⁺) and hydrolysable silicon associated with (especially, dispersed within and/or bound onto the surface of) one or more (solid or substantially solid; i.e., non-hollow) lipid globules, wherein the API is associated with (especially, bound to) the particles.

The optionally present lipid globules may have a mean diameter in a range of from about 50 nm to about 500 nm, especially about 50 nm to about 300 nm, more especially about 100 nm to about 300 nm.

API may be bound non-covalently to particles comprising one or more metals (especially, calcium; most especially, Ca²⁺) and hydrolysable silicon that are bound to the surface of one or more lipid globules. Up to about 10 or 20% of the API may be bound non-covalently to particles that are, in turn, bound to the surface of one or more lipid globules.

API may be bound non-covalently to particles comprising one or more metals (especially, calcium; most especially, Ca²⁺) and hydrolysable silicon that are, in turn, in the interior of one or more lipid globules. At least about 50, 60 or 70% of the API may preferably be bound non-covalently to particles that are in the interior of one or more lipid globules.

Preferably, API (especially, RNA, most especially mRNA) is bound non-covalently to particles comprising one or more metals (especially, calcium; most especially, Ca²⁺) and hydrolysable silicon that are in the interior of one or more lipid globules; and API (espeically, RNA, most especially mRNA) is bound non-covalently to particles that are bound to the surface of one or more lipid globules.

### Silicon Particle Aggregates

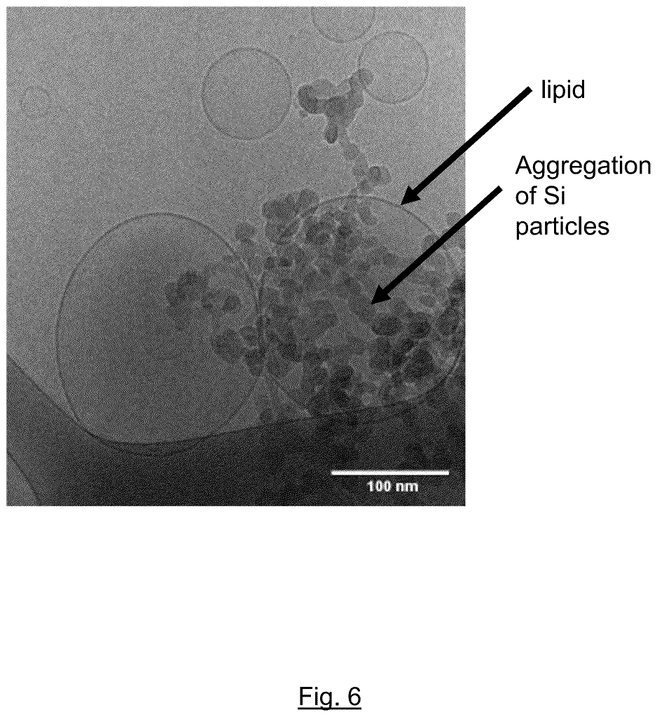

The particles comprising one or more metals (especially, calcium; most especially, Ca²⁺) and hydrolysable silicon may coalesce into aggregates of particles, for example as shown in the transmission electron microscope (TEM) image of FIG. 6.

Thus, the described composition may comprise aggregates (especially, chains) of the particles comprising one or more metals (especially, calcium; most especially, Ca²⁺) and hydrolysable silicon.

As used herein, the term “aggregate of particles comprising one or more metals and hydrolysable silicon” may refer to a cluster of particles wherein nearest-neighbour particles are in contact with each other. Such clusters may have varying configurations, such as substantially spherical clusters of particles and/or chains of particles. Particularly preferred are configurations comprising or consisting of chains of particles.

Thus, the composition may comprise one or more aggregates of particles comprising one or more metals and hydrolysable silicon. Preferably, the aggregates comprise one or more chains of the particles.

There may, for example, be present at least about 2, 3 or 4 chains on average per aggregate.

The one or more aggregates may comprise one or more branched chains of the particles. Thus, the one or more aggregates may be formed of, or may comprise, branches formed from chains of the particles. There may, for example, be present at least about 2, 3 or 4 branches per aggregate.

The one or more aggregates of particles may be associated with the one or more lipids, for example embedded in lipid and/or attached to the surface of lipid.

The one or more aggregates of particles may be associated with one or more lipid structures described herein, for example embedded within and/or attached to one or more lipid structures.

As described herein, the one or more lipid structures may be formed of or may comprise one or more of: micelles, incomplete micelles, liposomes, incomplete liposomes and lipid globules. Thus, the one or more aggregates of particles may be associated with (for example, embedded in and/or attached to the surface of) one or more of lipid micelles, incomplete lipid micelles, liposomes, incomplete liposomes and lipid globules.

In particular, the one or more aggregates of particles may be embedded in or attached to the surface of one or more of: liposomes; incomplete liposomes; and lipid globules.

The ratio of the longest dimension of an aggregate to the longest dimension of a lipid structure may on average be about 1:5 to 5:1, especially about 1:3 to 3:1; especially when the lipid structure is or comprises liposomes and/or lipid globules and the one or more aggregates are embedded therein or attached to the surface thereof. This may be measured, for example, by TEM as shown in FIG. 6.

The average longest dimension of an aggregate may be about 50 nm to about 500 nm, especially about 50 nm to about 200 nm, such as about 50 to about 150 nm, such as when measured by TEM. In particular, the one or more aggregates may be or comprise one or more chains of particles, with the average length of a chain being about 50 nm to about 500 nm, especially about 50 nm to about 200 nm, such as about 50 to about 150 nm. The average cross-sectional diameter of a chain may be about 5 nm to about 50 nm, such as about 5 nm to about 30 nm.

The ratio of the longest dimension of an individual particle to the longest dimension of a lipid structure may on average be in a range of from about 1:100 to about 1:2, especially about 1:100 to about 1:5, more especially about 1:100 to about 1:9; especially when the lipid structure is or comprises liposomes and/or lipid globules and the one or more aggregates are embedded therein or attached to the surface thereof. This may be measured, for example, by TEM as shown in FIG. 6.

When the one or more aggregates are present, the average (e.g., mean) diameter of a particle comprising the one or more metals and hydrolysable silicon may preferably be about 1 nm to about 50 nm, especially about 5 nm to about 50 nm, more especially about 1 nm to about 30 nm, yet more especially about 5 nm to about 20 nm, such as about 10 nm. Additionally or alternatively, the particles may be porous and may have an average (e.g., mean) pore diameter of about 0.1 to about 5 nm, such as about 2 nm.

In turn, API may be bound (non-covalently) to one or more of the particles in the aggregates.

Thus, the particles of the one or more aggregates may bind the API (especially, when the API is or comprises mRNA). When the one or more aggregates are or comprise chains of the particles, such chains may extend into the interior of the lipid structure(s) (especially, into liposomes, incomplete liposomes, and/or lipid globules). In this way, the chains may provide a route for the API to be better encapsulated in the lipid. Without wishing to be bound by theory, it is thought that this may shield the API from degradation, especially by shielding it from enzymes (espeically, in vivo) and preventing or reducing API molecules being available to react with water molecules. It is thought that as the silicon particles degrade over time, API may be released, thus enabling protection of the API until it reaches a target site for release.

The presence of charged API may itself facilitate aggregation of the particles. For example, when the API is nucleic acid (especially, mRNA) its negative charge (due to a phosphate backbone) may induce aggregation of the particles. Additionally or alternatively, the presence of Si—O species on the particles' surface may induce particle-particle interactions to facilitate aggregation.

Thus, preferably, the one or more aggregates are or comprise one or more chains of the particles, wherein API is bound to the particles, and the one or more chains extend into the interior of the lipid structure(s), especially, into liposomes, incomplete liposomes, or lipid globules. This may provide a stabler environment for the API. Additionally or alternatively, it may enable increased API uptake by the lipid structures, compared to no such aggregate(s) being present. This is in contrast to conventional liposomal delivery vehicles, for which inefficient API uptake may be a problem; for example, it is thought that up to about 80% of conventional liposomal delivery vehicles formulated into commercially available therapeutic compositions may be “empty” of API.

Optionally, substantially no particles are present as isolated particles; instead, substantially all particles are present in aggregates.

Aggregation may be promoted when the particles comprising one or more metals (especially, calcium; most especially, Ca²⁺) and hydrolysable silicon have an average diameter of about 1 nm to about 50 nm, especially about 5 nm to about 50 nm, more especially about 1 nm to about 30 nm, yet more especially about 5 nm to about 20 nm, such as about 10 nm. Such particle diameters may be below the typical diameter of lipidic structures (as described herein) that may form spontaneously (optionally, promoted by extrusion) from the one or more lipids.

Thus, preferably, the described composition comprises aggregates (especially, chains) of the particles comprising one or more metals (especially, calcium; most especially, Ca²⁺) and hydrolysable silicon, wherein the particles comprising have an average diameter of about 1 nm to about 50 nm, especially about 5 nm to about 50 nm, more especially about 1 nm to about 30 nm, yet more especially about 5 nm to about 20 nm, such as about 10 nm.

Aggregation may be promoted when the composition is extruded, for instance in the manner set out in the step “Extrusion” of “Materials and Methods” of Example 1 hereinbelow. Extrusion may be or comprise extrusion through a porous membrane having average pore diameters of about 0.01 μm to about 1 μm, such as about 0.05 μm to about 0.6 μm.

Accordingly, the composition may be (or be formed from) an extruded composition.

Preferably, extrusion occurs before addition of the API. Thus, the composition may be formed from an extruded composition comprising the particles and the one or more lipids, to which API has been added.

Therefore, the composition may suitably be an extruded composition that comprises aggregates (especially, chains) of the particles comprising one or more metals (especially, calcium; most especially, Ca²⁺) and hydrolysable silicon, wherein the particles have an average diameter of about 1 nm to about 50 nm, especially about 5 nm to about 50 nm, more especially about 1 nm to about 30 nm, yet more especially about 5 nm to about 20 nm, such as about 10 nm.

### Other Advantageous Components

Optionally, the composition further comprises one or more other advantageous components, especially one or more of tyrosine, NAD, quercetin and derivatives thereof.

The one or more other advantageous components may be or comprise tyrosine or a derivative thereof. Thus, optionally, in addition to the above-described amino acid, the specific amino acid tyrosine may be present as a further component. Optionally, the ratio by weight of the one or more lipids (i.e., total lipid components) to the tyrosine may be in a range of from about 130:1 to about 30:1, such as about 80:1.

Additionally or alternatively, the composition may comprise nicotinamide adenine dinucleotide (NAD) or a derivative thereof. Optionally, the ratio by weight of the one or more lipids (i.e., total lipid components) to the NAD may be in a range of from about 130:1 to about 30:1, such as about 80:1.

Additionally or alternatively, the composition may comprise quercetin or a derivative thereof. Optionally, the ratio by weight of the one or more lipids (i.e., total lipid components) to the as quercetin may be in a range of from about 130:1 to about 30:1, such as about 80:1.

The composition may optionally further comprise a peptide containing a cell surface receptor-(for example, integrin-) recognition sequence that confers a degree of cell specificity. The peptide may have a “head group” containing a cell surface receptor recognition sequence and additionally a “tail” that can bind non-covalently to the API (e.g., nucleic acid, such as mRNA) and/or the particles containing one or more metals and hydrolysable silicon.

Where the API is a nucleic acid, the composition may optionally further comprise a polycationic nucleic acid-binding component. The term “polycationic nucleic acid-binding component” is well known in the art and may refer to polymers having at least 3 repeats of cationic amino acid residues or other cationic unit bearing positively charged groups, such polymers being capable of complexion with a nucleic acid under physiological conditions. An example of a nucleic acid-binding polycationic molecule is an oligopeptide comprising one or more cationic amino acids. A polycationic nucleic acid-binding component may, for example, be an oligo-lysine molecule, an oligo-histidine molecule, an oligo-arginine molecule, an oligo-ornithine molecule, an oligo-diaminopropionic acid molecule, an oligo-diaminobutyric acid molecule, or a combined oligomer comprising or consisting of any combination of histidine, arginine, lysine, ornithine diaminopropionic acid, and diaminobutyric acid residues. Further examples of polycationic components include dendrimers and polyethylenimine.

Subjects and their Diseases and Disorders

According to preferred embodiments, the subject to whom the composition disclosed herein is administered, is a human subject. The age of the human subject may be in a range of from 1 month or above, optionally 1 year or above. The age of the human subject may be in a range of less than 100 years.

The disease or disorder in accordance with the present disclosure may be an infectious disease. As used herein, the term “infectious” may be used to refer to a disease which is liable to be transmitted from one organism to another, especially from one human to another.

The infectious disease may be a viral, bacterial, fungal, or parasitic disease; especially a viral disease.

Where the disease is a viral disease, it may be that of a respiratory virus, such, for example, as respiratory syncytial virus (RSV), parainfluenza virus (HPIV), metapneumovirus (HMPV), rhinovirus (HRV), coronavirus such as SARS-COV (especially SARS-COV-1, more especially SARS-COV-2), adenovirus (HAdV), enterovirus (EV), bocavirus (HBoV), parechovirus (HPeV) or an influenza virus.

The viral disease may be that of a dengue virus, Ebola virus, encephalomyocarditis virus, hepatitis virus, herpes virus, human immunodeficiency virus, human papillomavirus, human t-lymphotropic virus, measles virus, monkeypox virus, mumps virus, polio virus, rabies virus, rotavirus, rubella virus, varicella-zoster virus, west Nile virus, yellow fever virus or zika virus.

The disease or disorder may be a genetic disease or disorder.

In some embodiments, the genetic disorder may be characterised by a deficiency in the expression of one or more proteins, especially one or more enzymes.

The genetic disorder may be a multifactorial disorder, i.e. not confined to any specific pattern of single gene inheritance and likely to be associated with multiple genes effects together with the effects of environmental factors; such, for example, as schizophrenia, diabetes, asthma, depression, epilepsy, heart disease or hypothyroidism.

The genetic disorder may involve one or more mutations in one or more genes.

Thus, the genetic disorder may be a monogenic disorder, liable to occur if at least one mutation occurs in a single gene. Where the genetic disorder is a monogenic disorder, it may involve one mutation in the single gene or more than one mutation in the single gene. Examples of monogenic disorders include sickle cell anaemia, cystic fibrosis, Huntington's disease or Duchene muscular dystrophy.

The genetic disorder may involve one or more mutations in more than one gene. By way of non-limiting example, the genetic disorder may involve more than one mutation in a first gene and one mutation in a second gene.

The genetic disorder may be a disorder liable to occur if at least one mutation occurs in at least one gene amongst a set of genes; especially, such a genetic disorder may be osteopetrosis.

The genetic disorder may be Angelman syndrome; Canavan disease; Charcot-Marie-Tooth disease; colour blindness; cri du chat syndrome; cystic fibrosis; DiGeorge syndrome; Down Duchene syndrome; muscular dystrophy; familial hypercholesterolemia; haemochromatosis type 1; haemophilia; Klinefelter syndrome; neurofibromatosis; phenylketonuria; polycystic kidney disease; Prader-Willi syndrome; Scheuermann's disease; sickle cell disease; spinal muscular atrophy; Tay-Sachs disease; or Turner syndrome.

In its broadest sense, as used herein, the term genetic disorder may encompass cancer. The cancer may be or involve a blood cancer (such, for example, as a leukaemia, a lymphoma, or a myeloma) or a solid tumour (such, for example, as a sarcoma; a carcinoma; a carcinosarcoma; or a lymphoma).

Thus, especially, the cancer may be a cancer of the blood, skin, brain, prostate, breast, lung, oesophagus, stomach, small intestine, pancreas, colon and/or rectum, central nervous system, urinary bladder, thyroid, kidney, uterine corpus, oral cavity, or ovary.

In more detail, the cancer may be or involve a cancer of the pulmonary system, a brain cancer, a cancer of the gastrointestinal tract, a skin cancer, a genitourinary cancer, a pancreatic cancer, a lung cancer, a medulloblastoma, a basal cell carcinoma, a glioma, a breast cancer, a prostate cancer, a testicular cancer, an oesophageal cancer, a hepatocellular cancer, a gastric cancer, a gastrointestinal stromal tumour (GIST), a colon cancer, a colorectal cancer, an ovarian cancer, a melanoma, a neuroectodermal tumor, head and neck cancer, a sarcoma, a soft-tissue sarcoma, fibrosarcoma, myxosarcoma, liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, a leiomyosarcoma, a cervical cancer, a uterine cancer, an endometrial cancer, a carcinoma, a bladder carcinoma, an epithelial carcinoma, a squamous cell carcinoma, an adenocarcinoma, a bronchogenic carcinoma, a renal cell carcinoma, a hepatoma, a bile duct carcinoma, a neuroendocrine cancer, a carcinoid tumour, diffuse type giant cell tumour, or glioblastoma.

### Preparation, Storage, Stability and Administration of the Disclosed Compositions

Preventing a disease or disorder in a human subject may comprise administering a prophylactically effective amount of a composition disclosed herein to a human subject, wherein the subject is in need thereof, for example, identified by a physician or other healthcare practitioner as being in need thereof. Meanwhile, treating a disease or disorder in a human subject may comprise administering a therapeutically effective amount of a composition disclosed herein to a human subject in need thereof.

Dosage amounts of the composition disclosed herein may be varied so as to obtain an amount of the API which is effective to achieve the desired prophylactic and/or therapeutic response for a given subject, without being toxic to the subject. A suitable dosage amount of the composition may be the amount of the composition which is the lowest dosage amount effective for the API to produce a therapeutic and/or prophylactic effect.

The selected dosage amount, form and regime will each depend upon a variety of factors. Such factors may include, for example, the activity of the API, the route of administration, the time of administration, the rate of excretion or metabolism of the API, the rate and extent of absorption, the duration of the treatment, the presence of other drugs, compounds and/or materials used in combination with the API, the age, sex, weight, condition, general health and prior medical history of the subject being treated, and other such factors well known in the medical arts.

The composition may be administered by intramuscular or intravenous injection (encompassing transdermal delivery via a patch), orally (encompassing sublingual administration), intranasally, dermally, or by any other suitable route; especially by injection or orally.

Preferably, the composition may be administered by injection, such, for example, as intravenous or intramuscular injection. Optionally when the composition is administered by injection, the subject is monitored for symptoms or signs of a hypersensitivity response, such, for example, as a vaccine-associated hypersensitivity response.

Also preferably, the composition may be administered orally or intranasally. Compositions suitable for oral administration may be presented as discrete dosage forms, especially liquids or aerosol sprays each containing a predetermined amount of the composition. Such dosage forms may be prepared by any of the well-known methods of pharmacy.

The composition may be combined in an intimate admixture with a pharmaceutical carrier, according to conventional pharmaceutical compounding techniques. The carrier may take a wide variety of forms depending on the form of preparation desired for administration. Any of the usual pharmaceutical media may be employed as carriers, such as, for example, one or more of water, oils, and alcohols (encompassing glycols). The forms in which the disclosed compositions may be incorporated for administration, especially when formulated for administration by injection, orally or intranasally, may include aqueous solutions in saline. The composition may further include one or more pharmaceutically acceptable additives and excipients, such as one or more of the following: detackifiers, anti-foaming agents, buffering agents, polymers, antioxidants, chelating agents, viscomodulators, tonicifiers, odorants, opacifiers, suspending agents, fillers, plasticizers, flavouring agents, preservatives, colouring agents, diluents, binders, disintegrating agents and mixtures thereof.

The prevention or attenuation of the action of microorganisms may be brought about by the inclusion of various antibacterial and antifungal agents; such as one or more of the following: parabens, chlorobutanol, phenol, sorbic acid, thimerosal, and the like.

The composition disclosed herein may be provided in a sterile solution, by incorporating the composition in the required amount in an appropriate solvent (with various other ingredients, where appropriate) by any of the well-known methods of pharmacy. The composition disclosed herein may be provided in a sterile dispersion, by incorporating the composition in the required amount in an appropriate sterile vehicle (with various other ingredients, where appropriate). The composition disclosed herein may be provided as a sterile powder (e.g., for the subsequent preparation of sterile injectable solutions), such as by vacuum-drying and freeze-drying (lyophilisation) techniques which yield a powder of the composition.

Optionally, the composition may be stored before being administered to the subject. The composition may be stored at a temperature in a range of 0° C. or above, especially 4° C. or above, for a period of at least 1 week (optionally, up to 6 months, especially up to 1 year) prior to administering the composition to the subject.

In certain preferred embodiments, the particles, comprising one or more metals and hydrolysable silicon, and the one or more lipids are stored together but without the API. Shortly before administration to the subject, such as not more than about 3 weeks, about 2 weeks or about 1 week, especially not more than about 2 days, more especially not more than about 1 day before administration to the subject, they will be combined with the API. Advantageously, the particles and lipid may be storage stable for an extended period of time, especially a period of several months, such as about 6 months or about 12 months; they may be stored in this way without appreciable degradation. When the API is a reactive API, especially mRNA, it may advantageously be synthesised in situ or synthesised off-site and delivered to a clinical setting at the last possible moment, then combined with the particles and lipid(s).

Nonetheless, it has also been found that an API can be stabilised by the particles comprising one or more metals and hydrolysable silicon, including that the API can be stabilised during storage. Thus, in some embodiments, the particles can increase the storage stability of the API, especially where the API is or comprises nucleic acid such as mRNA. In some embodiments, the particles can increase the stability at 25° C. of the API, especially where the API is or comprises nucleic acid such as mRNA.

Thus, optionally, the composition being stored comprises all of the following components: particles comprising one or more metals and hydrolysable silicon; one or more lipids; and an active pharmaceutical ingredient (API). If so, the composition, once formulated, may optionally be stored for only a short period, by which may be meant a period of up to about 3 weeks, about 2 weeks or about 1 week prior to administration to the subject.

In some embodiments, the particles can increase the stability of the API during circulation in vivo, especially where the API is or comprises nucleic acid such as mRNA. In some embodiments, the particles can protect the API from degradation, especially enzymatic degradation, especially where the API is or comprises nucleic acid such as mRNA. Thus, the compositions disclosed herein may mitigate or address the problem of how to ensure APIs reach cells once they have been administered to a patient, including how to stabilise APIs while they are circulating in the body.

Meanwhile, the disclosed composition comprising one or more metals and hydrolysable silicon may mitigate or meet the need for tissue or cell targeting, so that an API can be delivered to the correct cells.

Additionally or alternatively, once a target cell is reached, the disclosed composition may mitigate or solve the challenge of how to ensure efficient API uptake by the cell. For example, the disclosed composition may assist trafficking of the API from the exterior of the cell into the cytoplasm.

Following uptake of the API by a cell, the disclosed composition may mitigate or solve the problem of how to prevent the API degrading too quickly in the cytoplasm. It is thought that the particles can increase the stability of the API in the cytoplasm of a cell, especially stability against enzymatic degradation, especially where the API is or comprises nucleic acid such as mRNA.

### Examples

### Example 1a—Manufacture of Calcium- and Silicon-Containing Particles

Porous silicon wafers were broken into 1 cm² films. They were then attached to more robust silicon substrates before undergoing an ultrasonic wash in IPA for 20 minutes.

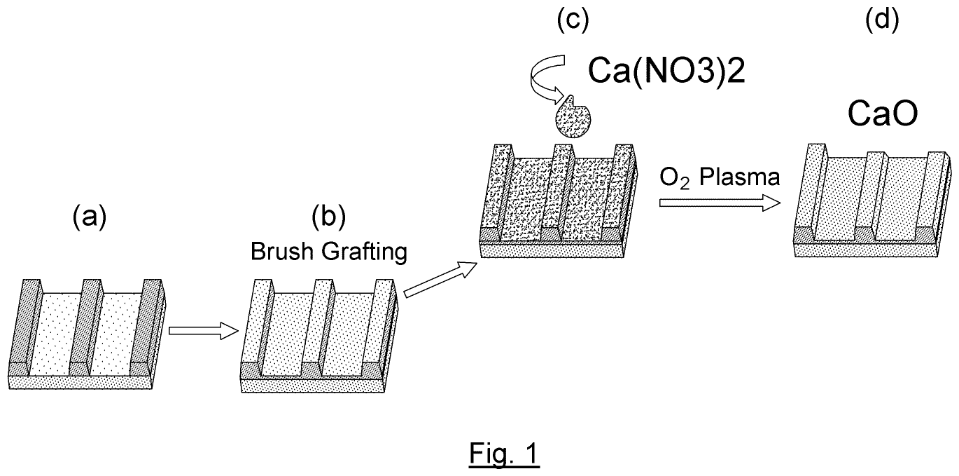

FIG. 1, part (a) provides an illustration of degreasing and hydroxyl functionalisation of the substrate surface, using oxygen plasma treatment for 2 minutes (60 s, 40 kHz, 50 W, Dianer PICO Barrel Asher). Polymers were dissolved in suitable solvents (PS—OH in toluene, PMMA-OH in toluene and P4VP in THF:IPA 3:2 mixture) based on Hansen solubility parameters and stirred overnight at 500 ppm to obtain homogeneous 0.2 wt. % solutions, which were then spin deposited onto the substrates at 3200 rpm for 30 seconds.

Thereafter, samples were placed on a hotplate at 230° C. for 2 minutes to form strong covalent bonding between the end hydroxyl group of the polymer and the complementary functional group present on the substrate through a condensation reaction, as shown in FIG. 1, part (b). Samples were then ultrasonicated in suitable solvents, in order to eliminate any physioabsorbed polymer, thus to yield chemically grafted monolayer films, as shown in FIG. 1, part (c). A monolayer polymer film was then infiltrated with 1 wt % ethanolic solution of calcium nitrate (Ca(NO₃)₂) and subsequently underwent oxygen plasma treatment of 1×10⁻² mbar of oxygen at 30 W for 20 minutes with an oxygen flow of 100 scem to oxidise the metal precursor and eliminate the polymer brush layer—as shown in FIG. 1, part (d)—thus forming a high quality metal oxide film.

This method enables manufacture nanoparticles (avg. diameter sub 30 nm) for SIS0015 (see below). Without wishing to be bound by theory, it is thought that Ca²⁺ ions are distributed throughout the Si matrix of these particles.

### Example 1b-Manufacture of Calcium- and Silicon-Containing Particles Via Calcium Salt Absorption

1 g silicon nanoparticles (avg. diameter sub 30 nm) are weighed into a beaker. The Si particles are then activated by suspension in 5 mL methanol. To this suspension is added 0.1 g of calcium chloride (CaCl₂)) powder. The suspension is left to dry overnight (as the methanol evaporates). The resultant particulate comprises both calcium and silicon.

This method enables manufacture of particles for SIS0014 (see below). Without wishing to be bound by theory, it is thought that Ca²⁺ ions are distributed on or close to the surface of these Si particles, in particular in a calcium silicate surface coating (and are not distributed throughout a hydrolysable silicon matrix).

### Example 1c—Manufacture of Magnesium- and Silicon-Containing Particles Via Magnesium Salt Absorption

1 g silicon nanoparticles (avg. diameter sub 30 nm) are weighed into a beaker. The Si particles are then activated by suspension in 5 mL methanol. To this suspension is added 0.1 g of magnesium sulphate (MgSO₄) powder. The suspension is left to dry overnight (as the methanol evaporates). The resultant particulate comprises both magnesium and silicon.

### Example 1d—Manufacture of Calcium-Containing Particles Via Calcium Ion Implantation

2 mm thick silicon discs having a 300 nm silicon oxide layer were polished on one side to mirror finish. The polished surfaces were then implanted with calcium ions at a fluence of 1×10¹⁷ Ca²⁺/cm², at an ion energy of 25 keV. Sample temperature during implantation did not exceed 403° C. Chamber pressure was approximately 10-6 Pa. The resultant particulate comprises both calcium and silicon.

This method enables manufacture of nanoparticles (avg. diameter sub 30 nm) for SIS0015 (see below).

### Example 1e—Manufacture of Calcium-Containing Particles Using Ca(NO₃)₂₄H₂O

250 mg cetrimonium bromide (CTAB; surfactant), 0.875 mL NaOH (2 M), and 116 mL of deionized water were mixed and heated to 70° C. under vigorous stirring. To this solution, 1.25 mL of tetraethyl orthosilicate was slowly injected, after which 2 mL of distilled water containing 0.938 g of Ca(NO₃)₂₄H₂O, and 0.146 mL of triethyl phosphate were subsequently added. After 3 h stirring, the silicon- and calcium-containing product was collected by centrifugation, washed with water and methanol, and dried under vacuum. The CTAB surfactant was removed from the pores by stirring in acidic methanol at 70° C. overnight, followed by centrifugation with washing with methanol and water, and drying under vacuum. The resultant particulate comprises both calcium and silicon.

This method enables manufacture of nanoparticles (avg. diameter sub 30 nm) for SIS0015 (see below).

### Example 1f—Zeta Potential Measurements

Particle zeta potentials were determined for the calcium- and silicon-containing samples, using dynamic light scattering (Malvern Zetasizer Pro (Red Advance), Malvern, UK).

It appears that when the silicon was surface functionalised with calcium, the zeta potential was somewhat different compared to where calcium was dispersed more deeply within the silicon structure. In both cases, the zeta potential was less negative than for pure Si, as shown in Table 1.

TABLE 1
Particle zeta potential

| Particle type | Zeta potential (mV) |
|---|---|
| Si only | −39 |
| Ca dispersed in hydrolysable Si matrix | −25 |
| of particles (including in the core) (for SIS0015) | |
| Ca on surface (not in the core) of | −31 |
| particles containing Si (for SIS0014) | |

### Example 2—Preparation of Particle and Lipid-Containing Compositions

Tables 2 to 4 indicate the components of the compositions prepared in Example 2.

TABLE 2
Composition of SIS0014 (having calcium- and silicon-containing nanoparticles)

| Lipids | | | Ca, Si - | | | |
|---|---|---|---|---|---|---|
| | | mPEG2000- | containing | | | Nuclease- |
| DOTAP | DOPE | DSPE | nanoparticles | Glycine | Trehalose | free water |
| 7.25 mg | 7.30 mg | 1.45 mg | 1 mg | 0.5 mg | 1 mg | Up to 10 mL |

| | | | | |
|---|---|---|---|---|
| 1.45 mL | 1.46 mL | 0.29 mL | 1 mL | Up to 10 mL |

TABLE 3
Composition of SIS0015 variants (having calcium-
and silicon-containing nanoparticles)

| | Ca, Si - | | | | | |
|---|---|---|---|---|---|---|
| | containing | | | | | Nuclease |
| | nanoparticles | Glycine | Trehalose | Tyrosine | Lipids | free water |
| SIS0015- | 1 mg | 0.5 mg | 1 mg | 0.2 mg | 16 mg | Up to 10 mL |
| T | 1 mL | | | 0.4 mL | | |
| | Ca, Si - | | | | | |
| | containing | | | | | Nuclease |
| | nanoparticles | Glycine | Trehalose | NAD | Lipids | free water |
| SIS0015- | 1 mg | 0.5 mg | 1 mg | 0.1 mg | 16 mg | Up to 10 mL |
| N | 1 mL | | | 0.2 mL | | |
| | Ca, Si - | | | | | |
| | containing | | | | | Nuclease |
| | nanoparticles | Glycine | Trehalose | Quercetin | Lipids | free water |
| SIS0015- | 1 mg | 0.5 mg | 1 mg | 0.2 mL | 16 mg | Up to 10 m |
| Q | | | | | | |

TABLE 4
Composition of SIS0028 (having magnesium-
and silicon-containing nanoparticles)

| | | | Activated | | | |
|---|---|---|---|---|---|---|
| Lipids | | | Mg | | | |
| | | mPEG2000- | absorbed | | | Nuclease- |
| DOTAP | DOPE | DSPE | SiNPs | Glycine | Trehalose | free water |
| 7.25 mg | 7.30 mg | 1.45 mg | 1 mg | 0.5 mg | 1 mg | Up to 10 mL |

| | | | | |
|---|---|---|---|---|
| 1.45 mL | 1.46 mL | 0.29 mL | 1 mL | Up to 10 mL |

The following protocol indicates how the compositions were prepared.

### Lipid Film Preparation

a) Mix all lipids in a glass round bottomed flask.
b) Evaporate any solvent present with rotary evaporator in water bath at 40° C. to form a lipid film on the inner surface of the flask.

### Rehydration of the Film

a) Add 1 ml of a suspension of the metal- and silicon-containing particles, together with glycine and trehalose (and, if present, tyrosine, quercetin or NAD), to the lipid film, along with 9 ml nuclease free water (total volume of the brownish suspension and nuclease free water together is thus 10 ml).
b) Cover the flask with parafilm, then agitate the flask in a water bath at 60° C. for 10 minutes, thus rehydrating the lipid film by means of the 10 ml of liquid.
c) Leave resultant suspension to rest at room temperature for a few hours before storing at 4° C.

### Extrusion

Pass the suspension obtained in step (c) of “Rehydration of the film” through a polycarbonate membrane filter having 0.4 μm and 0.1 μm pore sizes. Pass the suspension 10 times, at 60° C., through each pore size.

The resultant products are the compositions to be used in subsequent experiments.

### Example 3—Characterisation of the Compositions Prepared in Example 2

Particle sizes, polydispersity (PDI) and zeta potentials were determined using dynamic and electrophoretic light scattering (Malvern Zetasizer Pro (Red Advance), Malvern, UK). The results are shown in Table 5.

TABLE 5
Composition characterisation

| Sample | Size (nm) | PDI | Zeta Potential (mV) |
|---|---|---|---|
| SIS0014 (Ca on | 82.35 | 0.12 | +56.49 |
| surface (not in the | | | |
| core) of particles | | | |
| containing Si) | | | |
| SIS0015 (Ca dispersed | 89.1 | 0.14 | +57.37 |
| in the hydrolysable Si | | | |
| matrix of particles, | | | |
| including in the core) | | | |

SIS0015 has a more positive zeta potential than SIS0014. This may indicate an improved ability to modulate zeta potential when Ca²⁺ is dispersed in the hydrolysable Si matrix of particles, including in the core, as opposed to forming a coating of (it is thought) calcium silicate on the particles' surface. It may also be beneficial that for SIS0015, the zeta potential modulation by Ca²⁺ that has infiltrated to the particle core may be maintained as the particle shrinks/degrades, whereas this is not guaranteed for SIS0014 once the surface coating is lost.

### Example 4—Preparation of API-Loaded Compositions

For preparation of complexes with API (mRNA or siRNA), the relevant (SIS0014 or SIS0015) suspension, as obtained in Example 2, was mixed with siRNA or mRNA stock solution. The final concentration of siRNA or mRNA was adjusted using nuclease-free water, to obtain a ratio of total lipids to siRNA, or to mRNA, of 12:1.

The samples were mixed thoroughly by gently pipetting and were incubated at room temperature for 60 min to allow for complexation to complete. Following incubation, samples were stored at 4° C. prior to their use in subsequent assays.

### Example 5—Characterisation of siRNA-Loaded Compositions

Particle sizes, polydispersity (PDI) and zeta potentials were confirmed using dynamic and electrophoretic light scattering (Malvern Zetasizer Pro (Red Advance), Malvern, UK) for siRNA-containing compositions. The results are shown in Table 6.

TABLE 6
Composition characterisation

| Sample | Size (nm) | PDI | Zeta Potential (mV) |
|---|---|---|---|
| SIS0014 (Ca on | 134.4 | 0.24 | +40.27 |
| surface (not in the | | | |
| core) of particles | | | |
| containing Si) | | | |
| SIS0015 (Ca dispersed | 170.5 | 0.28 | +50.12 |
| in the hydrolysable Si | | | |
| matrix of particles, | | | |
| including in the core) | | | |

SIS0015 has a more positive zeta potential than SIS0014 when loaded with siRNA. This may indicate a particularly good modulation of nucleic acid binding stability for SIS0015 compared to SIS0014.

### Example 6—Transfection of Muscle Cells Using SIS0014 and Comparison to Si-Only Particles (SIS0012)

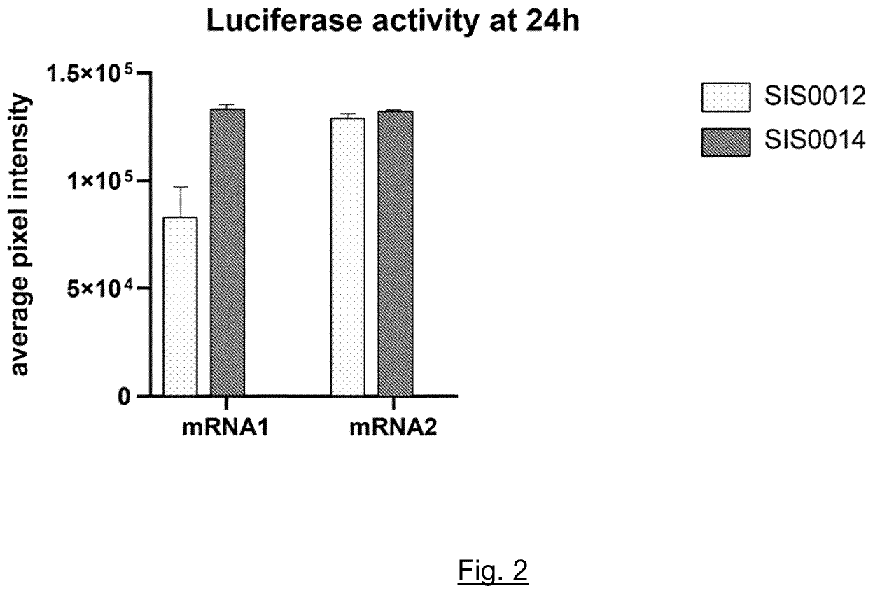

mRNA transfection by SIS0014 was investigated through in vitro transfection of rat skeletal muscle (L6C5) cells with luciferase-coding mRNA, observed by bioluminescence imaging of cell cultures. Bioluminescence was evaluated in triplicate at 24 hours post-transfection. A comparison was made to an equivalent composition containing Si-only particles (SIS0012). The results are shown in FIG. 2.

The details of the two luciferase-coding mRNAs used as test APIs are as follows.

(1) Firefly luciferase-coding mRNA having 2315 bases (mod-LUC RNA; 2 mg/mL) was obtained (referred to herein as “LUC mRNA1”, “Luc mRNA1” or simply “mRNA1”).

(2) Meanwhile, EZ Cap™ Firefly Luciferase mRNA having 1921 bases (modified by 5-moUTP), also luciferase-coding, was obtained (referred to herein as “LUC mRNA2”, “Luc mRNA2” or simply “mRNA2”).

For mRNA1, SIS0014 showed improved transfection than SIS0012, as evidenced by greater luciferase activity at 24 hours; for mRNA2, transfection was slightly better for SIS0014 than for SIS0012.

### Example 6—Transfection of Muscle Cells Using SIS0015 Variants and Comparison to Si-Only Variants (SIS0012)

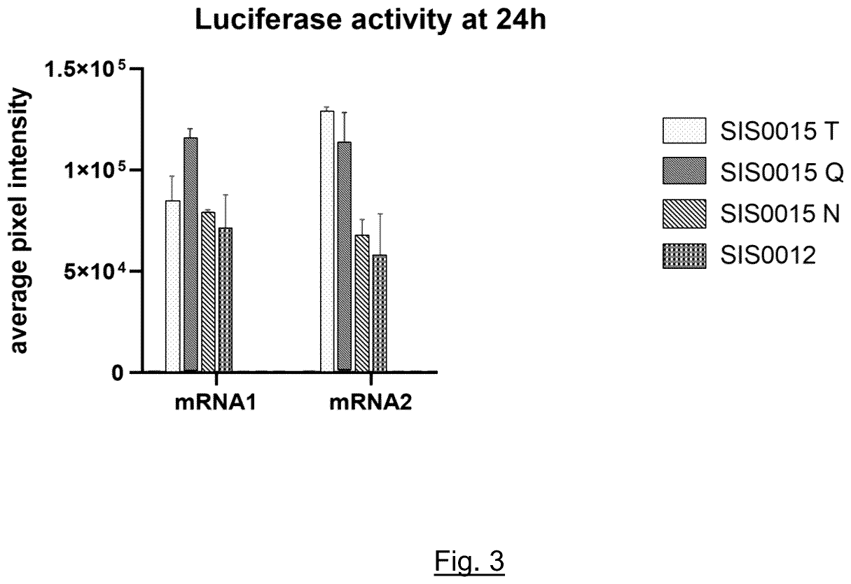

Example 5 was repeated, using SIS0015-T, -Q and -N (details given in Example 2 above) and comparing them to SIS0012. The conditions were otherwise the same as in Example 5. The results are shown in FIG. 3. SIS0015-T, -Q and -N show an improvement over SIS0012 for both mRNA1 and mRNA2, suggesting a more universal role in transfection improvement for SIS0015 compositions than for SIS0014 compositions.

### Example 7—Transfection of HEK293 Cells Using SIS0015 and its Variants

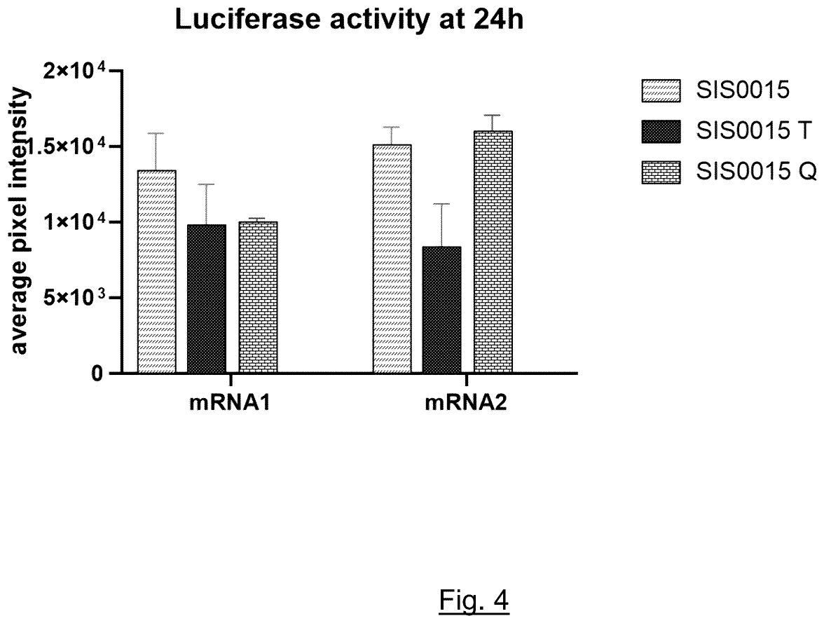

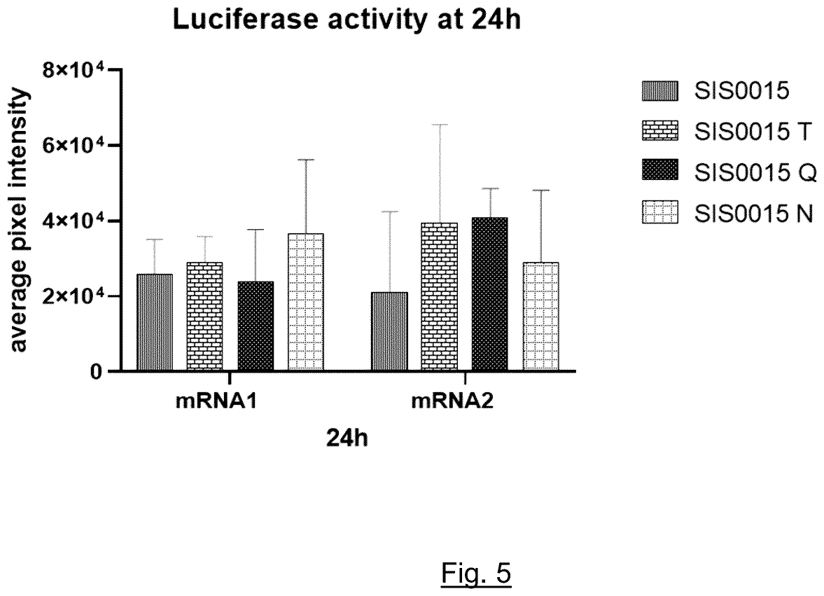

To review the suitability for transfection of different cell lines, Example 6 was repeated, using SIS0015, SIS0015-T and SSI0015-Q, in HEK293 cells. The conditions were otherwise the same as in Examples 5 and 6. The results are shown in FIG. 4. Error bars represent the standard deviation (calculated from three replicates). Adequate transfection efficiency was evidenced in this further cell line.

### Example 8—Transfection of L6C5 Cells Using SIS0015 and its Variants

To further review the suitability for transfection of different cell lines, Example 6 was repeated, using SIS0015, SIS0015-T, SSI0015-Q and SIS0015-N, in L6C5 cells. The conditions were otherwise the same as in Examples 5-7. The results are shown in FIG. 6. Error bars represent the standard deviation (calculated from three replicates). Adequate transfection efficiency was evidenced in this yet further cell line.

Where in the foregoing description, features or limitations are mentioned which have equivalents that are known, evident or foreseeable to those skilled in the art in the light of the present disclosure, then such equivalents are incorporated herein as if particularly set forth. Reference should be made primarily to the claims for determining the scope of the subject-matter of the present disclosure. The scope of protection sought by the present application further encompasses any such equivalents. It will also be appreciated by those skilled in the art that features or limitations of the disclosed subject-matter that are described as preferable, suitable, advantageous, convenient or the like may be optional and may not limit the scope of the independent claim(s) or the protection sought unless explicitly stated otherwise. Moreover, it is to be understood that such optional features or limitations, while of potential benefit in some implementations of the disclosed subject-matter, may be undesirable, and may therefore be absent or omitted in other implementations.

1. A composition comprising:
(i) particles comprising one or more metals and hydrolysable silicon;
(ii) one or more lipids; and
(iii) an active pharmaceutical ingredient (API).

2. A composition according to claim 1, wherein the particles comprising one or more metals and hydrolysable silicon contain at least about 50% by weight silicon atoms relative to the total weight of the particles.

3. A composition according to claim 1, the one or more metals comprising or consisting of one or more alkaline earth metals.

4. A composition according to claim 1, wherein, the metal is selected from the group consisting of: of lithium, sodium, potassium, magnesium and calcium.

5. (canceled)

6. (canceled)

7. (canceled)

8. A composition according to claim 1, wherein the one or more metals are present as cations.

9. A composition according to claim 8, wherein the cations are or comprise Ca²⁺.

10. (canceled)

11. (canceled)

12. A composition according to claim 1, wherein the one or more metals comprise metal atoms and/or metal ions in the cores of the particles.

13. A composition according to claim 12, wherein the metal atoms and/or metal ions in the cores are or comprise calcium.

14. A composition according to claim 1, wherein the API is zwitterionic or has a net negative charge at a pH of about 7.4.

15. A composition according to claim 1, wherein the API is or comprises a nucleic acid.

16. A composition according to claim 15, wherein the nucleic acid is RNA.

17. (canceled)

18. (canceled)

19. (canceled)

20. (canceled)

21. (canceled)

22. A composition according to claim 1, wherein the composition further comprises an amino acid.

23. A composition according to claim 1, wherein the composition further comprises one or more of tyrosine, NAD, quercetin and derivatives thereof.

24. (canceled)

25. (canceled)

26. (canceled)

27. (canceled)

28. A composition according to claim 1, wherein the composition further comprises a non-reducing disaccharide.

29. A composition according to claim 28, wherein the non-reducing disaccharide is trehalose.

30. A composition according to claim 1, wherein the one or more lipids comprise a lipid which is ionisable at a pH of about 7.4.

31. A composition according to claim 30, wherein the one or more lipids comprise a lipid which has a net positive charge at a pH of about 7.4.

32. A composition according to claim 1, wherein the one or more lipids comprise one or more lipidated oligopeptides.

33. (canceled)

34. (canceled)

35. (canceled)

36. A composition according to claim 1, wherein the particles increase the half-life in vivo of the API.

37. (canceled)

38. (canceled)

39. (canceled)

40. (canceled)

41. (canceled)

42. A composition according to claim 1, wherein the composition comprises one or more aggregates of the particles comprising one or more metals and hydrolysable silicon.

43. (canceled)

44. (canceled)

45. (canceled)

46. A composition according to claim 42, wherein mRNA is non-covalently bound to the particles in the one or more aggregates.

47. (canceled)

48. (canceled)

49. (canceled)

50. (canceled)

51. (canceled)

52. (canceled)

53. A method of preventing or treating a disease or disorder, comprising administering a prophylactically or therapeutically effective amount of a composition according to claim 1 as defined in to a human subject in need thereof.

54. (canceled)

55. (canceled)

56. A method for slowing the degradation of an API, comprising:
(i) contacting one or more lipids with particles comprising one or more metals and hydrolysable silicon; and
(ii) contacting the lipids and particles with the API.

57. A method according to claim 56, wherein the one or more metals comprise or consist of calcium.

58. A method according to claim 56, wherein the API comprises or consists of RNA.

59. (canceled)

60. (canceled)

61. A method according to claim 56, wherein the method results in increasing the half-life in vivo of the API.

62. (canceled)

63. (canceled)

64. (canceled)

65. (canceled)

66. (canceled)

67. (canceled)

68. A method according to claim 56, wherein the particles contain at least about 50% by weight silicon atoms relative to the total weight of the particles.

69. A method according to claim 56, further comprising contacting the lipids and particles obtained in step (i) with one or more of tyrosine, NAD, quercetin and derivatives thereof, prior to the step of contacting the lipids and particles with the API.
